(19) United States
(12) Patent Application Publication
(10) Pub. No.: US 2022/0177945 A1
(43) Pub. Date: Jun. 9, 2022

(54) METHOD FOR DETECTING AND ENUMERATING OF LOW CONCENTRATIONS OF LISTERIA

(71) Applicant: SENSILIST AS, KJELLER (NO)

(72) Inventors: Taran SKJERDAL, Hvalstad (NO); Tone MATHISEN FAGERENG, Ski (NO); Agnete FÆGRI, Oslo (NO); Isaac KOFITSYO SEWORNU CUDJOE, Knapstad (NO)

(21) Appl. No.: 17/416,822

(22) PCT Filed: Dec. 20, 2019
(86) PCT No.: PCT/EP2019/086742

§ 371 Date: Jun. 21, 2021

(30) Foreign Application Priority Data
Dec. 21, 2018 (NO) 20181688

(51) Int. Cl. C12Q 1/04; C12N 1/20; C12Q 1/08

(52) U.S. Cl. CPC C12Q 1/045; C12Q 1/08; C12N 1/205

(57) ABSTRACT

The present document is directed to a method for the detection and/or enumeration of Listeria bacteria, such as Listeria monocytogenes in a sample, said method comprising culturing a sample possibly containing Listeria bacteria in a culture medium comprising rhamnose, one or more of an antibiotic, a pH colour indicator and LiCl.

### TECHNICAL FIELD

The present document is related to methods and means for detecting and/or enumerating Listeria bacteria in a sample, such as Listeria monocytogenes, wherein the method comprises culturing samples possibly containing Listeria bacteria in a selective growth medium allowing a more rapid and sensitive detection of Listeria.

### BACKGROUND

Listeria monocytogenes is a food borne pathogen that causes thousands of listeriosis cases every year. In Europe alone, about 1500 cases are reported every year (EFSA 2018). Even though there are fewer listeriosis cases than of salmonellosis and campylobacteriosis, the fatality rate of listeriosis is higher.

Listeria monocytogenes is the species of pathogenic bacteria that causes the illness listeriosis. It is a gram-positive, facultative anaerobic bacterium capable of surviving in the presence or absence of oxygen. It can grow and reproduce inside the hosts cells and is one of the most virulent foodborne pathogens. Listeriosis infections in high-risk individuals may be fatal.

L. monocytogenes is a ubiquitous bacterium, which means that it is present in many different niches like soil, water, food, food production environments, animals and humans. It easily form reservoirs in food production facilities, even in factories with high hygienic standards and implemented HACCP systems. EFSA has reported a prevalence of L. monocytogenes of 0.5-6% in ready-to-eat foods in specific categories. The prevalence is highest in fish and seafood products (EFSA 2018).

Listeria monocytogenes has the ability to grow at temperatures as low as 0° C. and even down to −1.5° C., which permits proliferation at typical refrigeration temperatures, which thus greatly increases its ability to evade control in human foodstuffs.

The most vulnerable people to become ill from Listeria, are especially the elderly, people with compromised immune systems and pregnant women (fetus). Even a low number of bacteria, right down to 100-1000 colony forming units (cfu) per gram, can cause disease in high-risk groups, and an infection can be fatal and in the case of pregnant women even cause abortion. Healthy people are normally not affected by Listeria monocytogenes, but high doses can result in mild symptoms that are usually harmless. During the last decades 20-50 cases of listeriosis were recorded annually in Norway. This is about as frequently as in the rest of Europe, when measured per number of inhabitants.

The consequences of listeriosis are very serious, at worst it can cause death, and social costs are very high. Listeria monocytogenes can infect the brain, spinal cord membranes and/or the bloodstream of the host.

The combination of the facts that listeriosis is a very serious disease for humans, that listeria in food occurs relatively frequently, and that Listeria monocytogenes in food can be kept below disease causing numbers with relatively simple means, causes a strong focus on Listeria monocytogenes in legislation and with customers, including buyers of Norwegian fish.

Listeria can be analysed qualitatively or quantitatively. The ISO methods 11290-1 (qualitative) and 11290-2 (quantitative) are methods which standard in the legislation. However, these methods have detection limits 1 cfu/25 grams and 10 cfu/g, respectively, which is not sufficiently nuanced for analyses of at the process steps where the contamination is likely to occur, for instance during fish slaughter and filleting before the product is sent to the market. People in the fish trade have described the limitations with the ISO methods like this: If the methods were for measuring blood pressure, the results would have been that there is a blood pressure, less than 1000. This information is not nuanced enough to be useful. In the same way, methods for measuring Listeria concentrations in the range between 0.04 and 10 cfu/g is needed.

Listeria is able to grow in food during chilled storage. How high concentrations that are reached when the consumers eat the food will depend on the product conditions, storage conditions, process conditions and the vulnerability of the consumer who eat. In some products, like heat treated meat which is intended used cold in salads or fish used as sushi or sashimi, the concentration can under reasonably foreseeable conditions reach levels which represent a food health risk for healthy consumers if the initial concentration is above 2 cfu/g, but for vulnerable consumers, the initial concentration needs to be less than 0.2 cfu/g. In other products, the concentration can be higher, but in most cases, the acceptable concentration from a food safety point of view is between 0.2 and 5 cfu/g. The ISO method does not distinguish these concentrations, as levels below 10 cfu/g can not be enumerated unless many plates are applied, the sample is filtered or a higher concentration of sample in the broth is obtained in other ways.

The ISO methods were recently revised (2017). The changes from the previous version (1997) were these:

The main changes, compared to ISO 11290102:1998, are the following.
The enumeration of Listeria monocytogenes has been modified as listed below.
Primary suspension with buffered peptone water, half-Fraser broth with or without supplements, and all appropriate diluents referred to in ISO 6887 (all parts).
Resuscitation step deleted.
Microscopic aspect, catalase and CAMP test for confirmation are optional.
Inclusion of new performance characteristics
Moreover, enumeration of Listeria spp. has been included in the scope and the title changed accordingly.

In these revisions, enumeration of L. monocytogenes concentrations below 10 cfu/g have not, despite that the relevance was known, been addressed in the revision.

The qualitative ISO method 11290-1 includes a step in which Listeria receive optimum conditions to multiply, plating on selective agar medium and thereafter a confirmation step which qualitatively determines whether there are Listeria in the sample or not. However, with this qualitative method it is not possible to determine the actual number of bacteria in the sample and not even to distinguish between samples with a high or a low number of bacteria. This qualitative ISO method takes a week to perform after the sample has been received in the lab.

In the quantitative ISO method 11290-2, 10 grams of sample is analysed. One problem with this quantitative assay is that it has a detection limit of 10 cfu per gram, which is not sufficient. If the qualitative test is positive, but the quantitative negative, it is thus only possible to conclude that the product has less than 10 cfu Listeria per gram, it is however not possible to conclude if there are only for example 5 cfu or none at all.

The first step in the quantitative ISO method 11290-2 is plating a serial dilution of the sample to a selective growth medium, ALOA, and the second step involves confirmation on rhamnose, xylose, Gram and catalase testing. Lithium chloride is used as selective pressure in the ALOA medium and a colour indicator is used to indicate presumptive colonies. Confirmation with rhamnose and xylose is based on that only few bacteria can utilise these sugars. The quantitative ISO method thus comprises the following steps: cultivation of the sample on selective agar plate media for two days, and then confirmation. This ISO method takes 5 days to perform after the sample has been received in the lab.

Usually, a combination of the two above ISO methods is used. However, when it comes to the problem of Listeria in fresh salmon, a combination of these two methods will not be good enough, since it is not necessarily required that the fish must be completely free of Listeria to be regarded as safe. There may also be considerable variation within a product batch, and the samples taken are not necessarily representative of the whole batch.

One problem with the current methods is that the amount of sample that can be analysed is low, meaning that if the bacteria are unevenly distributed in the sample to be tested, bacteria may be missed and the sample evaluated as being negative for Listeria bacteria.

The food legislation in Europe has a food safety criterion for L. monocytogenes in ready-to-eat foods. The essence of this is that the L. monocytogenes concentration should not exceed 100 cfu/g at any time during the shelf life (EU regulation 2073/2005). Some countries have a zero tolerance of L. monocytogenes and at least partly due to the challenges in enumeration of low concentrations of Listeria, this option is discussed in several countries. However, the combination of 1) the relatively high prevalence of L. monocytogenes in food, 2) the challenge to remove L. monocytogenes from production facilities, and 3) the high concentrations before the likelihood of listeriosis increases all indicate that a zero tolerance is not realistic.

The 100 cfu/g concentration is considered as a reasonable trade-off between food safety and practical production. It is consensus for that this limit value is sufficient to avoid listeriosis cases, provided that the analytical method is sensitive and precise enough to detect and enumerate L. monocytogenes in the concentration range needed, and that the bacterium is sufficiently homogenously distributed in the batch so that the samples taken from the batch actually contain the bacterium in a representative concentration.

Unfortunately, none of these conditions are fulfilled. Studies with naturally contaminated food in commercial production have shown that only a few L. monocytogenes bacteria (typically 1 per 100 gram to 2 per gram) are transferred to the product during processing. These bacteria grow but remain local and form clusters in the food during storage. The sampling plan given in the microbial criterion in EU regulation 2073/2005 specifies 5-10 samples of 25 grams each, but it is questionable if this small sample size can cover the variability in terms of concentration. Secondly, the sampling of food is mainly carried out at the processing level, which means while the concentration of L. monocytogenes is far below 10 cfu per gram. The current reference ISO methods (ISO 11290-1 and 11290-2) have a detection level at 1 cfu/25 g in qualitative analysis and 10 cfu/g for enumeration. This means that a sample that is positive in qualitative analysis but negative in enumeration contains in the range 0.04-9.9 cfu per gram. This is a large range, resulting in that some food providers, customers and competent authorities have set the limit for withdrawal of foods at detection (0.04 cfu/g=1 cfu/25 gram) even though it leads to loss of food which represent a minimal risk. Others set the limit at 10 cfu/g even though these concentrations are too high to ensure that the 100 cfu/g limit by the end of shelf life is not exceeded.

Taken together, implementation of the food safety criterion for L. monocytogenes in ready-to-eat foods is challenging when sampling has to be done at the process level before the products are sent to the market, and more precise methods in the range 0.04-10 cfu/g are needed.

Thus there is a need for a more rapid and even more sensitive analysis which also can handle larger sample volumes for detecting Listeria, such as Listeria monocytogenes, in food samples as well as in swabs used to sample equipment surfaces.

An object of the present invention is thus to overcome or at least mitigate one or more of the problems described herein.

### SUMMARY

The present document is directed to a method for the detection of rhamnose fermenting Listeria spp., such as Listeria monocytogenes, in a sample, said method comprising or consisting of the steps of:
i) preparing a suspension of a sample potentially containing Listeria bacteria in a first culture medium, said first culture medium comprising rhamnose, one or more of an antibiotic, a pH colour indicator and LiCl;
ii) incubating said suspension at conditions allowing the growth of Listeria bacteria; and
iii) identifying positive samples

The method may further comprise the enumeration of rhamnose fermenting Listeria spp., wherein the method comprises a step ia) of transferring the suspension prepared in step i) to a multi-well tray and a step iv) performed after step iii) of calculating the concentration of said Listeria in said sample, such as by using the Most Probable Number method.

The method for enumeration of the number of rhamnose fermenting Listeria spp., such as Listeria monocytogenes, thus comprise or consists of the steps of:
i) preparing a suspension of a sample potentially containing Listeria bacteria in a first culture medium comprising rhamnose, one or more of an antibiotic, a pH colour indicator and LiCl;
ia) transferring the suspension obtained in step i) to a multi-well tray;
ii) incubating said suspension at conditions allowing the growth of Listeria bacteria;
iii) identifying positive samples; and
iv) calculating the concentration of said Listeria in said sample, such as by using the Most Probable Number method.

The method of the present document may further include a step of confirming the presence of Listeria monocytogenes by distinguishing Listeria monocytogenes from other Listeria species, such as by plating positive samples identified in step iii) on a second growth medium such as ALOA medium, by utilizing a molecular method, such as by polymerase chain reaction or in situ hybridization ELISA (enzyme-linked immunosorbent assay), VITEC or API (analytical profile index). This confirmation step is performed after step iii) in the qualitative method and after step iv) in the quantitative method.

The sample possibly containing rhamnose fermenting Listeria spp. may be processed by dividing said sample into small pieces such as by homogenization, slicing and/or chopping either before step i) is performed and/or in the first culture medium of step i).

The antibiotic may be one or more of nalidixic acid, ceftazidime, polymyxin B sulfate, cycloheximide, amphotericin B, preferably a combination of two or more of any of said antibiotics.

The pH colour indicator may be phenol red.

The LiCl may present in an amount of about 5-17 g/l, such as about 7-13 g/l, such as about 10 g/l.

The rhamnose may be present in an amount of about 5-17 g/l, such as about 7-13 g/l, such as about 10 g/l.

The sample may be a food sample, an environmental sample, or a sample from an animal, such as a human, such as a tissue sample or a feces sample.

The food sample may be raw or processed meat, poultry or fish products, vegetables or a ready-to-eat food product.

The environmental sample may be a water sample, a dirt sample or a food industry environmental sample, such as a surface swab sample.

The method may be performed in a closed system.

The present document is also directed to a culture medium for the growth and/or detection of rhamnose fermenting Listeria spp., such as Listeria monocytogenes, said culture medium comprising:
a) rhamnose in a concentration of about 5-15 g/1, such as about 7-13 g/l, such as about 10 g/l;
b) one or more antibiotics, such as nalidixic acid, ceftazidime, polymyxin B sulfate, cycloheximide, amphotericin B, preferably at least two of these;
c) a pH colour indicator, such as phenol red; and
d) LiCl in a concentration of about 5-17 g/l, such as about 7-13 g/l, such as about 10 g/l.

The present document is also directed to a kit for detecting and/or enumerating rhamnose fermenting Listeria spp, such as Listeria monocytogenes, in a sample, said kit comprising:
a) a container comprising a culture medium comprising or consisting of rhamnose, one or more of an antibiotic, a pH colour indicator and LiCl
b) a container or multi-well tray for cultivating a sample possibly containing Listeria bacteria;
c) optionally a colour chart for identifying positive samples; and
d) optionally instructions for use.

The concentrations of the constituents of the culture medium and the culture medium of the kit are the same as the concentrations of the constituents in the first culture medium described herein.

The present document also discloses a computer implemented calculator for displaying a prediction of the growth of Listeria, such as Listeria monocytogenes, based on the detection and enumeration of colony forming units in a sample by the method according to the present document, said calculator comprising:
a) an input device, such as a keyboard or a microphone;
a) an output device, such as a display, a computer or mobile phone screen or a loudspeaker.
b) software, downloadable or in memory, such as an app on a smart phone or a webpage;
   where the software accepts the input of any number of colony-forming units found in the sample in the range of 0.04 cfu/g to 1 cfu/g

Other features and advantages of the invention will be apparent from the following detailed description, drawings, examples, and from the claims.

### Definitions

Most Probable Number (MPN) is a method used to estimate the concentration of viable microorganisms in a sample by means of replicate liquid broth growth in x-fold dilutions (such as 15×, 10× or 2× dilutions). Most Probable Number may be used for estimating microbial populations in soils, waters, agricultural products and is particularly useful with samples that contain particulate material that interferes with plate count enumeration methods.

A colony-forming unit (cfu) is a unit used to estimate the number or viable bacteria or fungal cells in a sample. Viable is defined as the ability to multiply via binary fission under controlled conditions. Counting with colony-forming units requires culturing the microbes and counts only viable cells. The visual appearance of a colony in a cell culture requires significant growth.

“Cultivating”, “cultivation” and the like refers to growing or maintaining living cells in a growth or culture medium which is solid or liquid designed to support the growth of microorganisms or cells. The growth or culture medium comprises nutrients essential and relevant for the intended respective microorganisms or cells to grow.

ALOA (Agar medium for Listeria after Ottaviani and Augusti) medium is a selective chromogenic agar medium for the selective and differential isolation of Listeria monocytogenes. The medium is described in the ISO method.

Polymerase Chain Reaction, PCR, is a method of making multiple copies of a DNA sequence, involving repeated reactions with a polymerase.

In situ hybridization, ISH, is a technique that allows for precise localization of a specific segment of nucleic acid within a histologic section. The underlying basis of ISH is that nucleic acids, if preserved adequately within a histologic specimen, can be detected through the application of a complementary strand of nucleic acid to which a reporter molecule is attached.

By the term “first culture medium” is in the context of the present document intended a medium comprising rhamnose, one or more of an antibiotic, a pH colour indicator and LiCl. One example of a first culture medium is the “SensiList broth”, as disclosed elsewhere herein.

By “rhamnose fermenting Listeria spp” is herein intended Listeria spp that are able to ferment rhamnose. Examples of such bacteria include Listeria monocytogenes (L. monocytogenes), Listeria innocua (L. innocua) and Listeria welshimeri (L. welshimeri).

### BRIEF DESCRIPTION OF DRAWINGS

FIG. 1 shows an overview of the steps of the method disclosed in the present document. Left-handed track are the steps when the method is used qualitatively, while the right-handed track show the steps when the method is used quantitatively.

FIG. 2 shows the stacking pattern used when testing the sensitivity of the SensiList broth.

FIG. 3 shows the results of the testing of the sensitivity of the method of the present document.

FIG. 4 shows the results of the stability test of the SensiList broth.

FIG. 5 shows the results of a test performed in a company with L. monocytogenes in the production facilities. Samples 1, 3, 5 and 6 were production environment samples. No 3 and 5 turned yellow after incubation. Sample 2 and 4 were salmon samples, 100 g.

FIG. 6 shows the results of the analysis in Example 3.

FIG. 7 shows the results from a presumptive positive sample in Example 3.

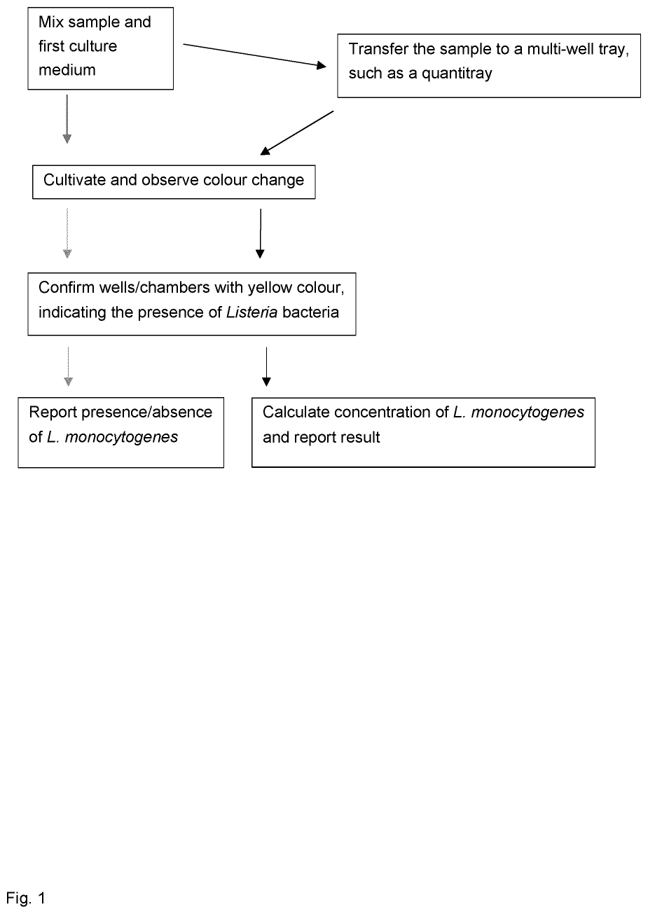

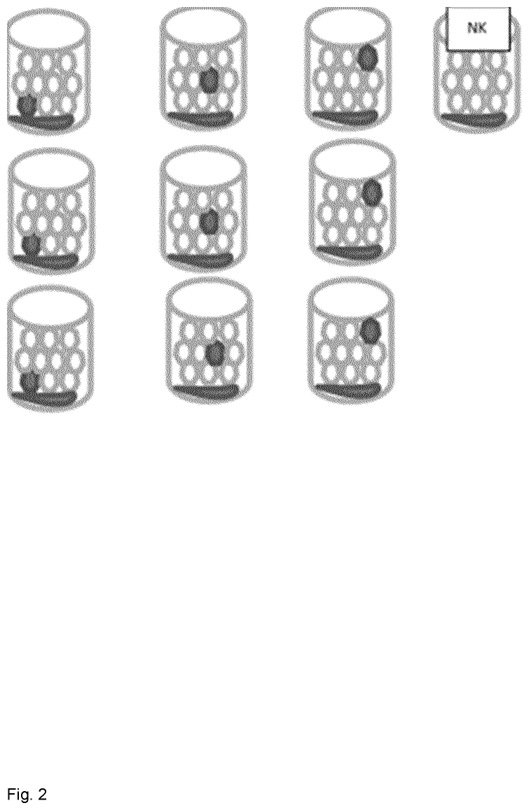

### DETAILED DESCRIPTION

The present inventors have developed a new method for the detection and/or enumeration of rhamnose fermenting Listeria spp. which has a high sensitivity and which is at least as accurate as existing methods, which is quicker to perform and which has a lower detection limit than the prevailing ISO methods. The method makes it possible to detect and enumerate very low numbers of rhamnose fermenting Listeria spp, such as Listeria monocytogenes and Listeria innocua in large or pooled samples, such as a food sample, such as salmon or chicken, as well as in surface swab samples.

The principle of the method of the present document is that a sample potentially comprising Listeria bacteria is inoculated in a culture (growth) medium which is selective for rhamnose fermenting Listeria spp, such as Listeria monocytogenes and Listeria innocua. Both these bacteria are able to ferment rhamnose to organic acids which lower the pH of the culture medium. The reaction is detected as a change in colour of the culture due to the presence of a pH indicator, such as phenol red, in the culture medium. A change in colour of the cultures is sufficient for detection of Listeria ssp, limited to L. monocytogenes and L. innocua. Confirmation studies can then be carried out to distinguish L. monocytogenes and L. innocua, such as by growing the bacteria on ALOA medium which is selective for L. monocytogenes. The culture medium further contains LiCl which allows a selective pressure for Listeria bacteria and antibiotics selected depending on the sample type and the bacteria not being Listeria known to be present in such samples.

The present document thus discloses a method for the detection of rhamnose fermenting Listeria spp, such as Listeria monocytogenes, in a sample, said method comprising or consisting of the steps of:
i) preparing a suspension of a sample potentially containing Listeria bacteria in a first culture medium, said first culture medium comprising rhamnose, one or more of an antibiotic, a pH colour indicator and LiCl;
ii) incubating said suspension at conditions allowing the growth of Listeria bacteria; and
iii) identifying positive samples (i.e. samples containing Listeria spp).

If a quantitative analysis of the number of rhamnose fermenting Listeria spp, in a sample is desired, the above method comprises the additional step ia) of transferring the suspension obtained in step i) to a multi-well tray and a step iv) of calculating the concentration of said Listeria spp. in said sample, such as by using the Most Probable Number method. A method for the detection and enumeration of Listeria in a sample thus comprises or consists of the steps of:
i) preparing a suspension of a sample potentially containing Listeria bacteria in a first culture medium comprising rhamnose, one or more of an antibiotic, a pH colour indicator and LiCl;
ia) transferring the suspension obtained in step i) to a multi-well tray;
ii) incubating said suspension at conditions allowing the growth of Listeria bacteria;
iii) identifying positive samples (i.e. samples containing Listeria spp); and
iv) calculating the concentration of said Listeria in said sample, such as by using the Most Probable Number method.

In order to confirm the presence of Listeria monocytogenes in positive samples identified in the above method, i.e. in samples containing rhamnose fermenting Listeria spp., the method comprises this confirmation as a further step performed after step ii) in the qualitative method and after step iv) in the qualitative method. The confirmation step may e.g. be performed by plating the positive samples on ALOA medium, by using a molecular method, such as PCR or in situ hybridization, ELISA. VITEC, and/or API.

The same method steps are basically used in the ISO 11290-1 and 2 methods, but in a different order and also with a different composition of the media used in the different steps which allows an integration of more than one step of the ISO methods in the same step in the present method, which leads to that the selectivity of the method of the present document is very similar to the ones of the ISO methods. However, the method of the present document has more selectivity built into the first step of the analysis than the ISO reference methods, which in turn leads to fewer presumptive positive samples and a more rapid identification of samples positive for Listeria bacteria, such as Listeria monocytogenes.

With the method of the present document, samples up to 125 grams can be analysed quantitatively with detection limit of 1 cfu/sample, i.e. a detection limit of 1 cfu/125 g and enumerated accurately in the concentration range of 1 cfu/25 grams up to about 2000 cfu/g. In the ISO 11290-1 and 11290-2 methods, the sample size is limited to maximum 25 g in the qualitative method and 10 g in the quantitative method. Further, the detection limit in the ISO methods is 10 cfu/g unless the method is modified to obtain a more concentrated sample. The selectivity and specificity of the method of the present document are as in the reference ISO methods used today (ISO 11290-1 and 11290-2), which means that the number of false negatives and positives in the method of the present document are as in the reference method.

The method of the present document is suited for analysis of foods (such as salmon and chicken) and swabs used to collect material from production facilities' surfaces. Furthermore, the method has the benefit that sampling and enumeration can be done in the production facilities, while confirmation and further characterization, for instance with whole genome sequencing, can be done at an external lab.

Another advantage of the method of the present document is that the sampling and sample preparation can be carried out by lay persons, ensuring that the food producer or auditor can take responsibility for this part themselves. Negative samples can be assessed locally while presumptive positive samples may be sent to a laboratory for confirmation.

The method disclosed herein can be used both qualitatively and quantitatively (by the addition of two method steps as described elsewhere herein and can be used for pooled samples so that one single analysis can be used to test compliance with the criterion in EU regulation 2073/2005. Pooled samples are in general more likely to give false negatives due to overgrowth of false positives, but this is not the case for the method of the present document if used quantitatively. In that case the solution is distributed in many wells which leads to that L. monocytogenes and false positives are not likely to be in the same chamber as if the whole solution was analysed at once. Thus, the presence of a step such as steps ia) and iv) described herein is a significant benefit since it allows for reducing the number of false positives.

The method of the present document ensures correct enumeration even if the sample has been exposed to abuse temperature conditions during transport to the lab. This is possible because the sample preparation and the start of the enumeration can be carried out together with the sampling. This is important, as the abuse temperature conditions between sampling in the facility and sample preparation in an external lab often lead to overestimation of the Listeria concentration in the food, and therefore unnecessary waste, recalls and incorrect decisions.

Compared to other rapid methods for Listeria detection, like InSite Listeria Test (Hyginia) Watford UK) and the Path-Chek Hygiene Listeria Test (Microgen Bioproducts Ltd., Camberley, UK), the false negative frequency is lower with the method according to the present document. (Bjørn C. T. Schirmer. Solveig Langsrud, Trond Møretrø, Therese Hagtvedt, Even Heir, 2012. Performance of two commercial rapid methods for sampling and detection of Listeria in small-scale cheese producing and salmon processing environments. Journal of Microbiological Methods, Volume 91, Issue 2, Pages 295-300, ISSN 0167-7012, https://doi.org/10.1016/j.mimet.2012.08.013.)

The ISO methods are given as standard in the legislation, and new methods need to be comparable with these methods on terms of specificity and other properties with relevance for validation of the method. For the users of analytical methods, the methods need to be cost effective and deliver results rapidly. This is particularly important if the results will imply on decisions for shipment, recalls, extra cleaning, etc. Therefore, an alternative method which fulfil these criteria, with the relevant selectivity and sensitivity is therefore needed.

The ISO methods consist of several steps. The first step is homogenisation of the sample in a broth, followed with cultivation in the broth (qualitative) or on agar plates with specialised growth medium (quantitative method). These growth media have been developed and optimised in several steps during the years in order to obtain
recovery of stressed cells in order to avoid false negatives.
increase selectivity by removing bacteria that can also grow on the media and cover Listeria on the agar plates.

The growth media in the ISO methods are suitable for enumeration down to 10 cfu/g, and are well suited in lab facilities where open systems like agar plates can be used. This is not the case in industry, where lower concentrations are needed and closed analytical systems must be used. Enumeration in broth with MPN (most probable number) methods is possible, but a way to read the difference between a positive and negative well is needed. The broth used for selective enrichment in the ISO method does not allow for that. For this reason, other growth media are needed if the enumeration of low levels with the MPN approach should be used.

Even though the growth media in the first steps in the ISO methods are suitable for detection and enumeration of L. monocytogenes down to 10 cfu/g, confirmation of presumptive positive colonies is needed. In one of the confirmation steps, rhamnose broth is used as L. monocytogenes is able to ferment rhamnose and produce acid which can be detected as a change in colour of a pH indicator. In the ISO method, there is no selective pressure in the rhamnose broth (which is used first at a later stage of the ISO methods), as only single colonies of isolates which, unlike bacteria in food, are already adapted to growth at the incubation temperature, are tested with this method. The broth does therefore not need to stimulate stress recovery.

In the method of the present document (the “SensiList” method), a modified version of the rhamnose broth as the primary growth medium is used and combined with the MPN method to allow enumeration of concentrations down to 0.2 cfu/g. To obtain this, the rhamnose broth has been designed and adapted in iterating steps to obtain:
Selectivity of L. monocytogenes (avoid false positive and negative bacteria)
Detection limit suitable for quantification in the range 0.2-100 cfu/g food material
Stress recovery of bacteria
Clear difference between positive and negative results
The system needs to be closed in order to ensure the biosecurity.
A price of the culture medium that makes the method cost effective.

There are more bacteria than Listeria which can utilise rhamnose. These should not be able to grow, at least not to the concentrations that will give a false positive signal in the method. Therefore, the antibiotics and other selective pressure components like LiCl which are used in the initial growth media in the ISO methods have been added to the rhamnose broth used in the initial enrichment step of the method of the present document.

The antibioticum/antibiotics used in the first culture medium are selected to reduce or avoid false positive results from other rhamnose fermenting bacteria. The specific antibiotics used may thus differ between different sample types. Microbes which can give false positive results can been searched for in databases and in commercial samples of food. The skilled person can therefore easily adapt the antibiotics used to the rhamnose fermenting bacteria that theoretically are present in that specific sample type. The antibioticum/antibiotics used in the first culture medium is/are thus specifically adapted to the sample type and any rhamnose fermenting non-Listeria bacteria that potentially are present in such a sample type

The list of antibiotics given in the recipe for the “SensiList” broth in this document are specifically adapted to fish samples.

Recovery of stressed cells involves that the cells need to have enough nutritious and good conditions to adapt its metabolism to growth modus. Listeria spp. are able to survive sub lethal conditions in a non-growing mode, but such mode can not be used to detect the bacterium. The components which cause selective pressure in the medium has therefore been tuned to an optimum between conditions for recovery and selectivity. Furthermore, rich medium components like meat extract has been added to the broth in low concentrations in order to supply nutrition for stress recovery. This is particularly important for samples which contain little organic material, like swab samples of production equipment and water.

The detection principle of the method of the present document is fermentation of rhamnose to acid which change the colour of a pH indicator. To obtain a sufficiently high production of acid, the bacteria need to use rhamnose as a nutrition and energy source, which in turn involves that the amount of other organic material in the broth, including sample, has to be low as the bacteria in otherwise would use this as nutrition and energy source. Furthermore, the amount of rhamnose has to be sufficiently high to allow production of enough bacteria and acid. The broth has been optimised and tested to balance these aspects. It has been found that both the amount of rhamnose and the concentration of rhamnose and eventually concentration of acid produced are critical for the detection of L. monocytogenes. The method has been optimised in order to ensure that all samples, even samples of swob from a water rich area, are correctly detected.

The method of the present document is in large based on the same principles as the ISO methods (ISO 11290-1 and 11290-2), but the order of the method steps is different and the media are modified. By performing these modifications, it was surprisingly found that the sample size could be increased while still maintaining and even increasing the sensitivity of the method, allowing a lower number of bacteria to be detected in a sample as compared to with the ISO methods. Further, the time from sampling until a result is achieved was substantially reduced.

The method of the present document differs from the ISO methods both regarding the steps that are used, the media that are used in the different steps, and the order of the steps. The below Tables 1 and 2 list some of the similarities and differences between the ISO methods and the method of the present document (Listeria monocytogenes is abbreviated with L. mono).

TABLE 1

| Criterion for analytical | ISO- | ISO- | SensiList- | SensiList- |
|---|---|---|---|---|
| method | qualitative | quantitative | qualitative | quantative |
| Specificity of Listeria | L. mono and | L. mono | L. mono and | L. mono and |
| spp and L. mono in the | Listera spp | Listeria spp | L. innocua | L. innocua |
| analytical step | | | | |
| Avoid false positive and | Gold | Gold | Comparable | Comparable |
| negative microbes | standard | standard | with ISO | with ISO |
| others than Listeria spp | | | | |
| Allow stress recovery | yes | yes | yes | yes |
| for the microbes in the | | | | |
| food | | | | |
| Sensitivity in the range | NA | no | NA | yes |
| 0.2-10 cfu/g | | | | |
| Detection limit for | yes | NA | Yes | NA |
| qualitative method 1 | | | | |
| cfu/25 gram or 1 | | | | |
| cfu/sample | | | | |
| Clear difference | ok | ok | ok | Ok |
| between positive and | | | | |
| negative results | | | | |
| Time until presumptive | 3 days | 2 days | 1-2 days | 1-2 days |
| positive result | | | | |
| Total time from | 5 | 5 | 2-3 | 2-3 |
| sampling to confirmed | | | | |
| positive result | | | | |
| Closed system | no | no | yes | yes |
| (assumption for use | | | | |
| outside of laboratory | | | | |
| facilities) | | | | |
| Possible to use in | no | no | yes | Yes |
| industry without lab | | | | |
| facilities | | | | |

TABLE 2

| Criterion | ISO | SensiList |
|---|---|---|
| Riktig - Correct | Identification: Yes | Identification: Yes |
| Relevant - relevant | Range for enumeration | Range for enumeration (0.2- |
| | (>10 cfu/g) not sensitive | 100) is the ideal area for |
| | enough at early process | implementation of |
| | steps | performance objectives in |
| | Relevant samples: food, | early process steps |
| | production environment, | Relevant samples: food, |
| | water | production environment, |
| | | water |
| Rask nok - sufficiently rapid | 2-3 days | 1-2 days |
| Robust - robust | Shelf life of broth: 3 months, | Shelf life of broth - ready to |
| | antibiotics delivered | use: 1 year |
| | separately | |
| Risikofri - no risk of leakage | No, open system | Yes, closed system |
| Renslig - minimal need of | No, homogenisation in open | Yes, homogenisation inside |
| cleaning, no mess. | system needed | closed system |
| Rimelig nok - reasonable | OK | Same range or cheaper |
| price | | than ISO |

In order to distinguish Listeria monocytogenes from Listeria innocua and other possible false positives due to other rhamnose fermenting bacteria, positive samples (i.e. yellow and orange wells if phenol red is used as a pH indicator) may be plated on ALOA medium and colonies with the characteristic zone indicate positive result for Listeria monocytogenes.

The method of the present document is faster than the ISO methods; it takes maximum two days for negative samples after sampling and three days until confirmed results if classical confirmation tests are used. Confirmation with PCR takes some hours, only. For comparison, the first step in the ISO method for enumeration (i.e. ISO 11290-2) is plating serial dilution of the sample to a selective growth medium, and the second step is confirmation on rhamnose. This method takes up to 5 The ISO method for detection (ISO 11290-1, detection level 1 cfu/25 g), has the following steps: cultivation of the sample in selective broth for two days, followed by plating on selective agar, and thereafter confirmation and takes a week after the sample has been received in the lab. Also, as a larger sample size can be used, it is e.g. possible to use pools of 10 samples, each 10 grams in one kit. This also leads to lower analysis costs for the food providers.

Culture Media

The first culture medium (an example of which is the herein so called “SensiList broth”) used in steps i)-iii) of the method of the present document is made selective for Listeria monocytogenes and Listeria innocua using three approaches.

Rhamnose is used as carbon and energy source in the first culture medium. Only few bacteria can utilize rhamnose, such as L. monocytogenes and L. innocua. L. weshimeri may also ferment rhamnose but is a very rare Listeria spp. in food compared to L. innocua and L. monocytogenes and even if this bacterium will grow in rhamnose containing medium, the specificity of the method of the present document is considered to be high enough and at least as high as for the presently available methods.

In the method of the present document, the selectivity of rhamnose is used already in the first step of enriching the bacteria (step ii) in the method of the present document).

Rhamnose is present in the first culture medium in a concentration of at least 5 g/l, such about 5-17 g/l, such as about 7-13 g/l, such as about 5 g/l, about 6 g/l, about 7 g/l, about 8 g/l, about 9 g/l, about 10 g/l, about 11 g/l, about 12 g/l, about 13 g/l, about 14 g/l, about 15 g/l, about 16 g/l, or about 17 g/l, typically about 10 g/l. Analytical grade rhamnose is an expensive component and as the volume of rhamnose broth needed in the method of the present document is high compared to in the ISO methods, the price of rhamnose is critical not only for the detection, but also for the price of the kit. Therefore, rhamnose food grade, which is significantly cheaper, has been tested for use in the present method and it was found that this quality of rhamnose gives the same results as the analytical grade, thus lowering the price for performing the method.

Lithium chloride (LiCl) is also added to the first culture medium. This component has been proved to increase the selective pressure in selective enrichment culture media for Listeria (including in the Fraser broth and ALOA plates that are used as a selective enrichment broth and diagnostic agar plate medium, respectively, in e.g. the ISO methods). Lithium chloride is present in an amount of at least about 5 g/l, such as about 5-17 g/l, such as about 7-13 g/l, such as about 5 g/l, about 6 g/l, about 7 g/l, about 8 g/l, about 9 g/l, about 10 g/l, about 11 g/l, about 12 g/l, about 13 g/l, about 14 g/l, about 15 g/l, about 16 g/l, or about 17 g/l, typically about 10 g/l.

Antibiotics are added in order to minimize growth of other Gram positive bacteria as explained elsewhere herein. Antibiotics suitable for use in the method of the present document include one or more of nalidixic acid, ceftazidime, polymyxin B sulfate, cycloheximide, amphotericin B. Preferably, a combination of two or more of any of said antibiotics is used, such as three, four, and even more preferably, all five are used in combination. The antibiotics are typically used in their normal concentrations, e.g. as specified in the recipe for the SensiList broth in the below. The need of antibiotics depend on the selection pressure needed for the sample to be analysed. Further, the same specificity is expected if the antibiotics used in the described first culture medium is exchanged with other antibiotics with similar modes of action.

By using the above three approaches, a selective pressure minimizing the growth of bacteria other than L. monocytogenes and L. innocua (and possibly other rhamnose fermenting bacteria present in the sample) is achieved. In order to allow a distinction between L. monocytogenes and L. innocua and other rhamnose fermenting bacteria, further confirmation is needed, such as plating on diagnostic agar, e.g. the ALOA agar, performing polymerase chain reaction or in situ hybridization analysis for specific L. monocytogenes genes, or by using a method such as ELISA, VITEC, API and/or performing a MaldiToff test.

In addition, the first culture medium may comprise organic carbon and energy sources in order to initiate rapid growth in phase and stress recovery. Sodium chloride may be added in order to obtain preferred osmotic conditions for the bacteria.

The first culture medium also contains a pH indicator. Fermentation is a process that lowers the pH. Thus, when bacteria that are able to ferment rhamnose are present in the sample, these will ferment the rhamnose present in the first culture medium, which will lower the pH of the culture medium. This change in pH is detected by including a pH indicator in the first culture medium. A change of the colour of the culture during incubation of the sample in the first culture medium thus indicates the presence of rhamnose fermenting Listeria spp., e.g. Listeria monocytogenes and Listeria innocua, in the sample. A suitable pH indicator for use in the method of the present document is phenol red, which changes colour from red to yellow when Listeria bacteria are present in a sample, but any other pH indicator which change colour at a similar pH as phenol red can be used.

The first culture medium can be used as ready-made product, or be prepared from single components. The phenol red broth and rhamnose may be sterilized by autoclavation at 121° C. The antibiotics are sterile filtered and added after autoclavation.

The first culture medium can be prepared in different ways such as:
mixing all components to a ready-to-use culture medium (if autoclavation is used for sterilizing the medium, this is done before addition of the antibiotics (which typically are filter sterilized)
preparing rhamnose and phenol red broth base as one solution, and adding the antibiotics just shortly before use, but after autoclavation. Antibiotics are generally less stable than other components in growth media, and late addition is therefore a way to extend the shelf life of the culture medium, and to minimize the probability of false positives,
a concentrated culture medium with the rhamnose and phenol red broth (such as 5-15× concentration, e.g. 10× concentrated) can be prepared, and diluted with water before use.

This requires sterile filtration of the culture medium instead of autoclaving due to caramellisation reactions of the medium.

The first culture medium is preferably liquid.

In all cases, the broth can be placed directly in a kit, or separately in flasks or bags in larger volumes.

The first culture medium comprises or consists of
a) rhamnose in a concentration of about 5-15 g/l, such as about 7-13 g/l, such as about 10 g/l;
b) one or more antibiotics, such as nalidixic acid, ceftazidime, polymyxin B sulfate, cycloheximide, amphotericin B, preferably at least two of these;
c) a pH colour indicator, such as phenol red; and
d) LiCl in a concentration of about from 5 to about 15 g/l, such as about from 7 to about 13 g/l, such as about 10 g/l.

One example of a first culture medium providing a good selective pressure is given below. This medium is herein denoted “SensiList broth”:

SensiList Broth Recipe:

Preparation of the 1% Rhamnose Medium:

| | | |
|---|---|---|
| Phenol red broth base | 15 | g |
| Rhamnose | 10 | g |
| LiCl | 10 | g |
| Destilled water | 1000 | ml |

Dissolve the components in distilled water. Sterilize for 15 min at 118° C. Cool down before adding antibiotics.

Preparation of the Antibiotic:

| | | |
|---|---|---|
| Nalidixin acid | 10 | mg/ml |
| Polymyxin B | 200 000 | IE/ml |
| Amphotericin B | 2 | mg/ml |
| Ceftazidime | 2 | mg/ml |

Dissolve the antibiotics in distilled water to the specified concentrations. Sterilize by sterile filtration.

Completion of the SensiList Broth:

| | | |
|---|---|---|
| 1% Rhamnose medium | 1 | L |
| Nalidixin acid (10 mg/ml) | 2 | ml |
| Polymyxin B (200,000 IE/ml) | 0.3835 | ml |
| Amphotericin B (2 mg/ml) | 5 | ml |
| Ceftazidime (2 mg/ml) | 4 | ml |

Add the antibiotics to the 1% Rhamnose dilution. Mix well.

pH: 7.4±0.2

The SensiList broth is particularly useful for the detection and enumeration of Listeria in fish samples.

Due to the presence of both rhamnose and LiCl (in combination with antibiotic(s) a higher selective pressure for Listeria bacteria in the first cultivation step ii) in the method of the present document is obtained than in the quantitative and qualitative ISO methods, which use two cultivation steps to achieve the same result.

In order to confirm the presence/absence of Listeria monocytogenes in samples identified as positive for the presence of rhamnose fermenting Listeria spp. in step iii) in the method of the present document, the positive samples can be plated on a second growth medium, such as ALOA medium, which allows L. monocytogenes to be distinguished from L. innocua in that the former forms clear precipitation zones while the latter does not. ALOA medium contains a specific purified substrate for the enzyme phosphatidylinositol-specific phospholipase C which allows for the distinction between L. monocytogenes and L. innocua as the former forms an opaque halo around the colonies when metabolizing the substrate. The ALOA medium is preferably a solid medium, such as an agar medium.

Samples and Sample Preparation

A sample can be any sample that is subject for examination of the presence or absence of Listeria bacteria, such as Listeria monocytogenes. The sample can e.g. be a food sample, an environmental sample, or a faeces sample.

A food sample can be e.g. a sample of raw food or a sample of processed meat, poultry or fish products such as samples of salmon, vegetables or a ready-to eat food product. The environmental sample can be a water sample e.g. from a bleeding tank, thawing tank, washing water, sea water, a dirt sample or a food industry environmental sample, such as a surface swab sample or sample equipment surfaces.

Food product samples, typically in the size of 10-125 grams, may be added to e.g. buffered peptone water (BPW) or physiological saline in the ratio 1:1 and homogenized in a stomacher bag, either by hand or in a stomacher before being transferred to the first culture medium, e.g. in a ratio of homogenized sample to culture medium of 1:10. Alternatively, the sample can be homogenized in the first culture medium directly.

Environmental swab samples, such as cloths potentially containing Listeria bacteria (either dry or with a buffer added) can be added directly to the first culture medium, and left there during incubation. The procedure is otherwise as for food samples.

Water samples, such as process waters, including washing water, chilling water, bleeding water, etc., can be added to the first culture medium. As for food product samples, a concentrated first culture medium can be used in order to lower the detection level and at the same time limit the volume and thereby space in the incubator. For instance, 11 ml of a 10× concentrated first culture medium can be added to a 100 ml water sample in order to obtain a 1 cfu/100 ml detection limit. The procedure for incubation and detection of presumptive positive samples are the same as for food samples.

Method for Detection and/or Enumeration

As mentioned above, for all different kinds of samples the method disclosed herein can be performed qualitatively or quantitatively depending on which steps are included in it.

The method for detection (i.e. a qualitative method) of rhamnose fermenting Listeria spp., such as Listeria monocytogenes, in a sample, comprises or consists of the steps of:
iv) preparing a suspension of a sample potentially containing Listeria bacteria in a first culture medium, said first culture medium comprising rhamnose, one or more of an antibiotic, a pH colour indicator and LiCl;
v) incubating said suspension at conditions allowing the growth of Listeria bacteria; and
vi) identifying positive samples

As mentioned above, the method may further comprise the enumeration of rhamnose fermenting Listeria spp., wherein the method comprises a step ia) of transferring the suspension prepared in step i) to a multi-well tray and a step iv) performed after step iii) of calculating the concentration of said Listeria in said sample, such as by using the Most Probable Number method.

The method for enumeration (i.e. a quantitative method) of the number of rhamnose fermenting Listeria spp., such as Listeria monocytogenes, thus comprise or consists of the steps of:
i) preparing a suspension of a sample potentially containing Listeria bacteria in a first culture medium comprising rhamnose, one or more of an antibiotic, a pH colour indicator and LiCl;
ia) transferring the suspension obtained in step i) to a multi-well tray;
ii) incubating said suspension at conditions allowing the growth of Listeria bacteria;
iii) identifying positive samples; and
iv) calculating the concentration of said Listeria in said sample, such as by using the Most Probable Number method.

The method of the present document may further include a step of confirming the presence of Listeria monocytogenes by distinguishing Listeria monocytogenes from other Listeria species, such as by plating positive samples identified in step iii) on a second growth medium such as ALOA medium, by utilizing a molecular method, such as by polymerase chain reaction or in situ hybridization, ELISA, VITEC, and/or API. This step is performed after step iii) in the qualitative method and after step iv) of the quantitative method.

For both qualitative and quantitative analyses of the presence of Listeria bacteria in a sample, the first step in the method is the preparation of a suspension of a sample possibly containing Listeria bacteria in the first culture medium, as described elsewhere herein.

The sample(s) is/are typically incubated at a temperature of about 37° C., even if temperatures such as between 25 and 38° C. may also be used, such as a temperature of about 30° C., and the colour of the culture(s) observed after 1 and 2 days. A change in colour, such as from red to yellow or orange when phenol red is used as a pH indicator, indicates a presumptive positive sample. The colour can be read as such, or a colour code be found by comparing with a colour chart.

If a qualitative method is to be performed, it is sufficient to incubate the suspension of a sample possibly containing rhamnose fermenting Listeria spp. to observe the presence or absence of a colour change during incubation of the sample in the first culture medium (i.e. the suspension of a sample potentially containing rhamnose fermenting Listeria spp. in the first culture medium). If a colour change is observed, rhamnose fermenting Listeria spp., such as. Listeria monocytogenes or Listeria innocua were present in the sample, and the sample is thus positive for rhamnose fermenting Listeria spp. If no colour change is observed, rhamnose fermenting Listeria spp. were absent in the sample, i.e. the sample is negative for Listeria bacteria.

For a quantitative method to be performed to enumerate the concentration of rhamnose fermenting Listeria spp. the original sample, the suspension of a sample possibly containing rhamnose fermenting Listeria spp. in the first culture medium is transferred to a multi-well tray (plate), such as a quantitray, such as Quanti-Tray 2000 from IDEXX. During incubation under conditions such as described above, wells containing rhamnose fermenting Listeria spp. will change colour due to the presence of the pH indicator in the first culture medium. The most likely concentration of rhamnose fermenting Listeria spp. in the original sample can then be estimated using the MPN (Most Probable Number) method. For example, if a quantitray is used for estimation of the concentration of rhamnose fermenting Listeria spp. in a sample, and the total volume of the suspension of a sample potentially containing rhamnose fermenting Listeria spp. sample is 100 ml, and the sample size is 5 g, the detection limit will be 1 cfu/5 g samples, which corresponds to an average concentration of 0.2 cfu/g sample. If the concentration of rhamnose fermenting Listeria spp. is high, for instance 100 cfu/g, there will be 500 bacteria in 100 ml solution containing 5 grams of sample. This corresponds to 5 cfu/ml. It is then likely that most of the 1 ml wells in the quantitray turn yellow, while maximum 50% of the 0.1 ml wells turn yellow. With the double concentration of rhamnose fermenting Listeria spp., up to 100% of the +0.1 ml wells will turn yellow. Above this concentration, all wells will be yellow, and the upper limit for valid enumeration is reached. The minimum and maximum limit for enumeration using a quantitray of 96 1 ml and 96 0.1 ml wells will then be in the range 1 cfu/5 gram (i.e. 0.2 cfu/g) to 200 cfu/gram. If another ratio between sample and the first culture medium is applied, the detection limit can be adapted.

The method of the present document makes it possible to detect and enumerate extremely low levels of Listeria monocytogenes and rhamnose fermenting Listeria spp. in large or pooled samples. The sample size can be as high as 100 grams and the detection level as low as 5 cfu/100 g food, i.e. 1 cfu/20 g food. As the sample size is large, it is possible to use pools of e.g. 10 samples, each 10 grams, in one kit. This also leads to lower analysis costs for the food providers. Also, the larger possible sample size, enabling large or pooled samples, is an important factor in order to overcome issues due to the uneven distribution of Listeria in food products.

Analyses performed with the method of the present document can be performed in the production facility, because the system may be made closed so that the enriched Listeria positive samples are not able to leak bacteria back to the production facilities or to the persons handling the samples. Closing the system means that after addition of the sample to the first culture medium, independently of whether the method is quantitative or qualitative, the container (such as a flask, bag, beaker, multitray etc.) is sealed so that the bacteria potentially growing in the medium does not get in contact with air, people handling the samples or surfaces touching the sample. The sample preparation, like homogenisation and cutting, can be done inside the closed system. No gas exchange is needed, as Listeria can grow without oxygen. Closing the system may e.g. be achieved by sealing the sample, e.g with a lid or a plastic film. e.g. in a manner that only allows opening of the system with a specific tool, etc. However, confirmation of presumptive positive samples for the presence of L. monocytogenes has to be carried out in a laboratory classified for Listeria analyses, because the container with broth needs to be opened.

All kinds of samples can be shipped to the lab for confirmation/testing of the presence of L. monocytogenes. If the sample is a presumptive positive one, i.e. the colour has turned to yellow after incubation of the sample in the first culture medium, the temperature should be kept low during shipping in order to maintain the bacteria. In case the sample is sent shortly after it has been placed in the first culture medium, no chilling is desired, as growth during shipping shorten the detection time after the sample has reached the lab.

Several methods can be used for confirmation of L. monocytogenes:
Forming of zones on ALOA agar. L. monocytogenes form zones, while L. innocua does not.
PCR analysis for detection of L. monocytogenes. Actually, this test can be performed before the sample turn yellow, as the number of cells before a positive PCR reaction is obtained is reached before the cell number is so high the pH go below the level for colour change to yellow.
in situ hybridization analysis for specific L. monocytogenes genes,
Any other identification method, for instance ELISA, VITEC, API, MaldiToff test and whole genome sequencing.

As is evident from the above, the method of the present document allows a presumptive positive and negative results to be achieved in one to two days both for the qualitative and the quantitative method, and confirmed positive results maximum one day later, as opposed to the ISO methods' 5 or 7 days (for the quantitative and the qualitative method, respectively. Further, an advantage of the present method is that it does not have to be performed in a lab, but can be performed e.g. at the site the sample is taken, such as in the industry itself.

Kit

The present document is also directed to a kit for the detection of Listeria, such as Listeria monocytogenes, in a sample, said kit comprising:
a) a container comprising a culture medium comprising or consisting of rhamnose, one or more of an antibiotic, a pH colour indicator and LiCl
b) a container or multi-well tray for cultivating a sample possibly containing Listeria bacteria;
c) optionally a colour chart for identifying positive samples; and
d) optionally instructions for use.

The concentrations of the constituents of the culture medium in the kit are as disclosed elsewhere herein for the first culture medium. The first culture medium is preferably liquid although it may also be solid.

Computer Implemented Calculator

The present document is also directed to a computer implemented calculator for displaying a prediction of the growth of Listeria, such as Listeria monocytogenes, based on the detection and enumeration of colony forming units in a sample, said calculator comprising:
a) an input device, such as a keyboard or a microphone
a) an output device, such as a display, a computer or mobile phone screen or a loudspeaker
b) software, downloadable or in memory, such as an app on a smart phone or a webpage

With the input device, the number of colony-forming units is set to any number found in the sample, in the range 0.04 cfu/g to 10 cfu/g, and especially in the range 0.04 cfu/g to 1 cfu/g. Prior art has not allowed for such details in the low range.

The calculator may have inputs for parameters such as the pH of the sample or the type of sample, for example raw salmon, raw fish, sushi, chicken or beef. In addition, there are inputs for such parameters as temperature and storage time in production, shop and in the home fridge as well as the room temperature and the number of hours outside refrigeration. Based on the input, the calculator will calculate from stored models and then display with graphs or numbers various prediction such as the number of days for highest growth, most likely growth, least possible growth and plot these versus the legal limit, the limit for possible disease with exposed consumers and the limit for possible disease with and healthy adult.

The calculator may be implemented as a single application per type of food, or as an integrated calculator with different choices for the user.

The invention will be further described in the following examples, which do not limit the scope of the invention described in the claims.

### EXPERIMENTAL SECTION

Experimental Section

### 1 Example 1: Development and Testing of the SensiList Broth

1.1 Sensilist Broth

The contents of the culture medium in the below referred to as “Sensilist broth” described above.

1.2 Sensitivity of the Sensilist Broth

The first studies of selectivity and sensitivity of the SensiList broth were performed with salmon samples taken directly from a production company. Salmon pieces were inoculated with either 0, 2, 20 or 200 cfu/g of Listeria monocytogenes. As inoculum, either single strains or a cocktail of 5 strains was used. The samples were used as either immediately, or stored frozen until use.

The concentration of LiCl and rhamnose in the SensiList broth was tuned in these studies.

The samples were analysed both with the SensiList and the ISO methods in order to compare the results. The ISO method was adapted in order to allow lower detection levels in two ways. Firstly, a larger volume of the sample suspension was plated by using five times more plates. This approach is not feasible in daily routine, but was applied here in order to verify the sensitivity of the method of the present document. Secondly, a 5× concentration of the sample suspension was used by adding less diluent before plating on the agar medium as described in the ISO method.

Analysis with the method of the present document: The salmon samples were prepared as described below (product samples) and placed in SensiList broth. For qualitative samples (presence/absence), the samples and broth were placed in 100 or 500 ml flasks. For enumeration with the MPN (most probable number) method, the broth with sample was placed in sterile glass tubes (1-10 ml) or in Quantitray 2000 from IDEEXX with 49 large wells and 24 small wells, altogether having a volume of 100 ml. The broth was incubated as described below, and the colour of the broth observed after one and two days. All wells were further analysed by transferring a droplet to ALOA plates, using a sterile needle. Bluegreen colonies with typical zones were presumptive positive for L. monocytogenes, bluegreen colonies without zones were presumptive positive for L. innocua. Colonies with other colours were negative. The bluegreen colonies were confirmed with Gram staining and catalase, according to ISO 11290-1.

The studies were repeated about 10 times in triplicate with salmon inoculated with 2, 20 or 200 cfu/g of L. monocytogenes. In some experiments, one Listeria positive sample (cube approx. 2×2×2 cm) was placed together with 9 negative samples of the same size and stacked in different formations, as shown in FIG. 2. The purpose was to investigate whether positive samples were detected for the three sampling procedures i) analysis of the all samples in the stack, ii) analysis of the broth after shaking the samples with broth, followed by incubation of the broth, and iii) analysis of released water from fish.

1.3 Selectivity of the SensiList Broth

False positive and negative bacteria were searched for in two ways:
1. Strains used as positive and negative control strains in Listeria diagnostics and in testing of kits were selected.
2 Salmon from different suppliers were purchased from shops in the Oslo area, and cultivated in SensiList broth without antibiotics and LiCl in order to allow growth of all bacteria which were able to utilize rhamnose. Tubes where the broth changed colour from red to yellow during the incubation were plated on blood agar and ALOA agar, and bacteria isolated. The isolates were used in later experiments to 1) formulate the antibiotic composition in the SensiList broth and 2) test the stability of the broth.

1.4 Confirmation of Presumptive Positive Sample with PCR

Confirmation with PCR was tested with these methods:

The DNeasy® Blood & Tissue kit (Qiagen) was used to extract DNA from Listeria monocytogenes (strain VI 58361, serotype 1/2a) cultivated in growth medium broths. These growth media were used: SensiList broth, SensiList broth without LiCl and antibiotics, SensiList broth without antibiotics. In addition, Half Fraser medium and buffered peptone water were included as controls. A colony of L. monocytogenes was transferred from agar plates to the different broths. DNA was extracted with the kit according to the procedure from the supplier, and the DNA concentration measured using Nanodrop (spectrophotometer from ThermoFischer for measuring DNA concentrations).

The isolated DNA was analysed with qPCR (quantitative PCR) according for the following genetic markers: ORF2819, ORF2110, Imo1118, Imo0737, pIcA and prs using the BioRad iQ-Check® Listeria monocytogenes II kit for both DNA extraction and the PCR.

1.5 Stability of the SensiList Broth

The stability of different formulations of the SensiList broth was checked as follows: Four formulations of the SensiList broth were prepared in sufficiently large quanta to perform analyses several times during one year of storage. The formulations used were SensiList broth without LiCl and antibiotics, the broth with LiCl but no antibiotics, the broth with antibiotics added immediately before use, and the full broth. The experiments were repeated with three independent batches of each formulation.

The experiments were performed using single strains in the groups L. monocytogenes (8 strains), other Listeria spp (2 strains), other bacteria which may give false positive results. The latter strains were selected based on own experiments with salmon (see above), strains used to test the ISO method, and strains that give false positive results in other kits for L. monocytogenes. The strains are described in more detail below.
A. Listeria monocytogenes strains:
   a. 2 strains used for testing of the ALOA medium in the ISO-method.
      i. L. monocytogenes serovar 4b VI 60847 (WDCM00021/ATCC 13932)
      ii. L. monocytogenes serovar 1/2a, VI 51285 (WDCM00109/CCUG 15527)
B. L. monocytogenes strains used in the experiments with Sensilist broth:
   a. VI 51503
   b. VI 58363 (00EB250LM)
   c. VI 58365 (00EB254LM)
   d. VI 59994
   e. VI 59998
   f. VI 58386
C. Listeria innocua: used for testing of the ALOA medium in the ISO method.
   a. VI 51284 (WDCM00017/CCUG 15531)
D. Listeria ivanovii: used for testing of the ALOA medium in the ISO-method.
   a. VI 51040 (ATCC 19119)
E. Other strains:
   a. Used for testing of ALOA:
      i. Enterococcus faecalis (VI 52179).
      ii. E. coli VI 51656 (WDCM00013/CCUG 17620)
   b. C. albinans V106652 (ATCC10231)
F. 18 strains from salmon giving false positive results in early versions of the SensiList when antibiotics were not applied
   a. 5 Hafnia alvei: VI 54910, VI 54914. VI 54918, VI 54924, VI 54927
   b. 5 Citrobacter sp: VI 54915, VI 54916, VI 54928, VI 54931, VI 54940
   c. 2 Aeromonas sp: VI 54922, VI 54926
   d. 2 Enterobacter cloacae: VI 54935. VI 54941
   e. 1 Alcaligenes faecalis: VI 54929
   f. 1 Enterococcus faecalis: VI 54946
   g. 1 Bacillus cereus: VI 54948
   h. 1 usikker identifisering, kanskje en Macrococcus: VI 54952
G. 2 strains giving false positives in other kits
   a. Carnobacterium maltaromaticum: VI 61406
   b. Enterococcus casseliflavus: VI 61407
H. Strains which are able to ferment rhamnose:
   a. Lactobacillus rhamnosus: VI 60846

L. monocytogenes strains were cultivated and cold-adapted to 7° C. for 7 days, in order to adapt them to representative stress condition for food samples. The other strains, which could give false positive samples were cultivated over night at 37° C. and transferred to BHI medium. The different temperatures was applied in order to prepare a worst case scenario: the L. monocytogenes strains should be given the realistic stress in order to detect false negative results, while the false positive strains should be given the best possible options to grow in the broth during incubation.

The formulations of SensiList broth (0.9 ml) were inoculated (0.1 ml) in 96 well boxes (type) This mix was diluted tenfold in 8 steps in the tray, leading to one column per strain, as illustrated for tray 1 below. Three trays were needed per medium formulation to test all the strains. The boxes were covered before incubation at 37° C., and the colour changes observed after 1 and 2 days after incubation.

TABLE 3
Distribution of the strains used in the stability test in 96-wells plates (12 columns and 8 rows in each plate). Each
column contains 1 strain that was diluted 10-fold. Row A contains the first 10 fold diluted strains, row B contain
the second 10 fold dilution and so on. The 5 letter numbers on the top is the identification of each strain.

| | A | | | | | | | | | | | |
|---|---|---|---|---|---|---|---|---|---|---|---|---|
| | Listeria monocytogenes | | | | | | | L. inn | L. ivan | Ent. faec | E. coli | C. albi |
| | VI | | | | | | | | | | | |
| | 58366 | 51285 | 51503 | 58363 | 58365 | 59994 | 59998 | 51284 | 51040 | 52179 | 51656 | 06652 |
| | 1 | 2 | 3 | 4 | 5 | 6 | 7 | 8 | 9 | 10 | 11 | 12 |
| A | 10⁻¹ | 10⁻¹ | 10⁻¹ | 10⁻¹ | 10⁻¹ | 10⁻¹ | 10⁻¹ | 10⁻¹ | 10⁻¹ | 10⁻¹ | 10⁻¹ | 10⁻¹ |
| B | 10⁻² | 10⁻² | 10⁻² | 10⁻² | 10⁻² | 10⁻² | 10⁻² | 10⁻² | 10⁻² | 10⁻² | 10⁻² | 10⁻² |
| C | 10⁻³ | 10⁻³ | 10⁻³ | 10⁻³ | 10⁻³ | 10⁻³ | 10⁻³ | 10⁻³ | 10⁻³ | 10⁻³ | 10⁻³ | 10⁻³ |
| D | 10⁻⁴ | 10⁻⁴ | 10⁻⁴ | 10⁻⁴ | 10⁻⁴ | 10⁻⁴ | 10⁻⁴ | 10⁻⁴ | 10⁻⁴ | 10⁻⁴ | 10⁻⁴ | 10⁻⁴ |
| E | 10⁻⁵ | 10⁻⁵ | 10⁻⁵ | 10⁻⁵ | 10⁻⁵ | 10⁻⁵ | 10⁻⁵ | 10⁻⁵ | 10⁻⁵ | 10⁻⁵ | 10⁻⁵ | 10⁻⁵ |
| F | 10⁻⁶ | 10⁻⁶ | 10⁻⁶ | 10⁻⁶ | 10⁻⁶ | 10⁻⁶ | 10⁻⁶ | 10⁻⁶ | 10⁻⁶ | 10⁻⁶ | 10⁻⁶ | 10⁻⁶ |
| G | 10⁻⁷ | 10⁻⁷ | 10⁻⁷ | 10⁻⁷ | 10⁻⁷ | 10⁻⁷ | 10⁻⁷ | 10⁻⁷ | 10⁻⁷ | 10⁻⁷ | 10⁻⁷ | 10⁻⁷ |
| H | 10⁻⁸ | 10⁻⁸ | 10⁻⁸ | 10⁻⁸ | 10⁻⁸ | 10⁻⁸ | 10⁻⁸ | 10⁻⁸ | 10⁻⁸ | 10⁻⁸ | 10⁻⁸ | 10⁻⁸ |

1.6 Treatment of Samples for Analysis with the Method of the Present Document

1.6.1 Food Product Samples

The samples, 10-125 grams, were added to buffered peptone water (BPW) in the ratio 1:1 and homogenized in a bag, either by hand or in a stomacher. 10 ml of the homogenized solution were added 90 ml of SensiList broth. The entire sample was incubated at 37° C. and the colour observed after 1 and 2 days. A change from red to yellow solution indicates a presumptive positive sample. The colour can be read as such, or a colour code be found by comparing with a colour chart. For example, a positive result could be indicated by using the colour chart from www.qioco.no colour Y50R.

The test solution contains 5 grams of product, if prepared as described above. The entire solution is included, which leads to a detection level of 1 cfu/5 grams. If another detection level is desired, this can be obtained if another ratio between sample and broth is applied. Some examples are given below. If a large sample is desired, for instance a pooled sample of 5*25 gram sample in order to fulfil the microbial criterion absence in 25 grams, measured in 5 samples, the total volume can be limited by adding the broth as a concentrated solution. For instance, 125 gram sample+125 ml BPW+50 ml SensiList broth in 6× concentration limits the total volume to 300 ml. This is a sufficient high liquid fraction to observe a colour change, but a higher dilution factor will be needed for some foods in quantitative analysis.

TABLE 4
Examples of different ratios of broth to sample
in view of the desired detection level.

| | Procedure | Comments |
|---|---|---|
| 1 cfu/25 gram (i.e. | Add a mixture of 50 ml | In the quality method |
| 0.04 cfu/g) | sample + BPW to a | from ISO and NMKL the |
| | bottle of 450 SensiList | detection limit is |
| | broth. | 1 cfu/25 g. |
| 1 cfu/5 gram (i.e. | 1 bottle as described | |
| 002 cfu/g) | above. | |
| 1 cfu/2 gram (i.e. | Add 4 ml of sample + | |
| 0.5 cfu/g) | BPW to a bottle of 90 ml | |
| | SensiList broth. | |
| 2 cfu/g | Add 1 ml of sample + | This limit is crucial |
| | BPW to a bottle of 90 ml | if the fish is intended |
| | SensiList broth. | to be eaten raw, |
| | | according the results |
| | | from BASELINE. |

1.6.2 Swabs from Production Environment Samples

Swabs, being cloths of various kinds, dry or added a buffer, can be added directly to the SensiList broth, and left in the container during incubation. The procedure is otherwise as for food products.

1.6.3 Water Samples

Process waters, including washing water, chilling water, bleeding water, etc., can be added to SensiList broth. As for product samples, a concentrated broth can be used in order to lower the detection level and at the same time limit the volume and thereby space in the incubator. For instance, a 100 ml water sample can be added 11 ml of a 10× SensiList broth in order to obtain a 1 cfu/100 ml detection limit. The procedure for incubation and detection of presumptive positive samples are the same as for food samples.

1.6.4 Method for Enumeration—Quantitative Analyses

All categories of samples can be performed qualitatively or quantitatively. To enumerate the Listeria concentrations in the samples, the liquid fractions of the SensiList broth solutions described above are transferred to e.g. a quantitray, where the broth is separated in 96 small and large wells. If the entire volume (100 ml) contains 1 Listeria bacterium, one of the wells will turn yellow while the others remain red. The most likely concentration can be estimated using the MPN method. In the case 100 ml solution contains 5 grams of sample, the detection limit will be 1 cfu/5 g samples, which corresponds to an average concentration of 0.2 cfu/g sample. If the concentration of Listeria is high, for instance 100 cfu/g, there will be 500 bacteria in 100 ml solution containing 5 grams of sample. This corresponds to 5 cfu/ml. It is then likely that all 1 ml wells turn yellow, while maximum 50% of the 0.1 ml wells turn yellow. With the double concentration of Listeria, up to 100% of the +0.1 ml wells will turn yellow. Above this concentration, all wells will be yellow, and the upper limit for valid enumeration is reached. The minimum and maximum limit for enumeration in a quantitray of 96 1 ml and 96 0.1 ml wells will then be in the range 1 cfu/5 gram to 200 cfu/gram.

The detection limit can be tuned by using another ratio between sample and SensiList broth.

1.7 Tests of the Method of the Present Document with Artificially and Naturally Contaminated Samples

1.7.1 Case 1: Artificially and Naturally Contaminated Salmon

The released thawing water of inoculated samples were analysed with the SensiList and modified ISO method. The procedure is described above and the purpose of the study in the result chapter.

1.7.2 Case 2: Contaminated Heat Treated Chicken Meat

The samples were inoculated with low concentrations of L. monocytogenes and analysed according to the food sample protocol described above.

1.7.3 Case 3: Naturally Contaminated Meat and Milk Products. Processed Products from Several Animal Species

The samples were analysed according to the food sample protocol described above.

1.74 Case 4: Swabs for Testing of Production Surfaces after Cleaning

The samples were inoculated and analysed according to the protocol for environment samples described above.

1.7.5 Case 5: Test in Production Company

SensiList broth in flasks were sent to a salmon slaughter and filleting facility. A short instruction note was and a colour map was also provided. The company took samples according to their normal procedure, added them to the SensiList broth and incubated them at 37° C. After incubation, the company took pictures and sent them to us in order to discuss the results. They also sent the samples to their local lab for confirmation.

1.8 Results

1.9 Development of the Method

1.9.1 Accuracy and Sensitivity of the Method—Detection Level

There was a good correlation between inoculated concentration, concentration enumerated with the method of the present document and ISO method for the concentrations 200 and 20 cfu/g. For salmon with 2 cfu/g, L. monocytogenes were detected only with the method of the present document. This is as expected, as the detection level for the ISO method is 10 cfu/g when performed according to the standard procedure. For all the concentrations, the presumptive positive concentrations were confirmed.

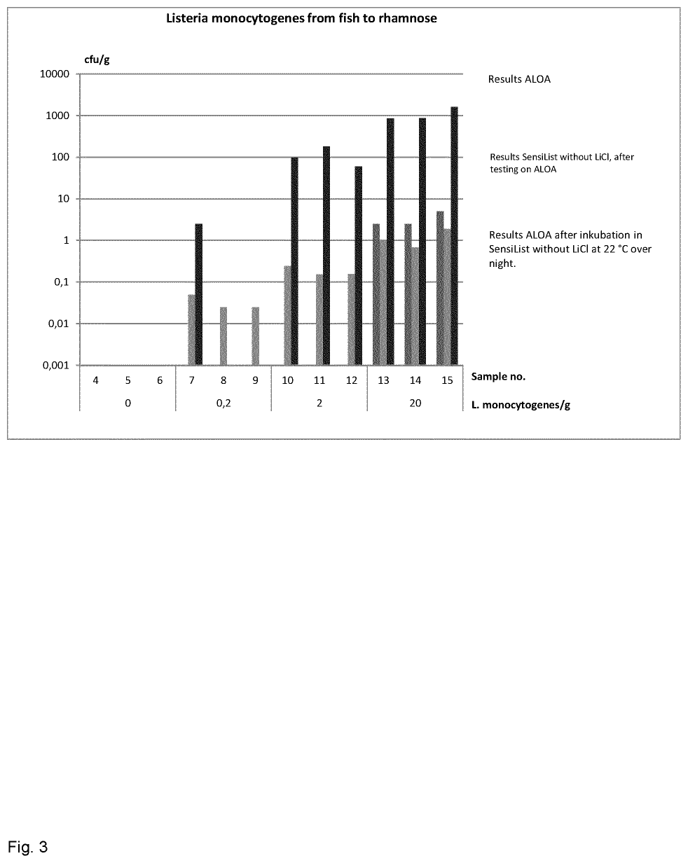

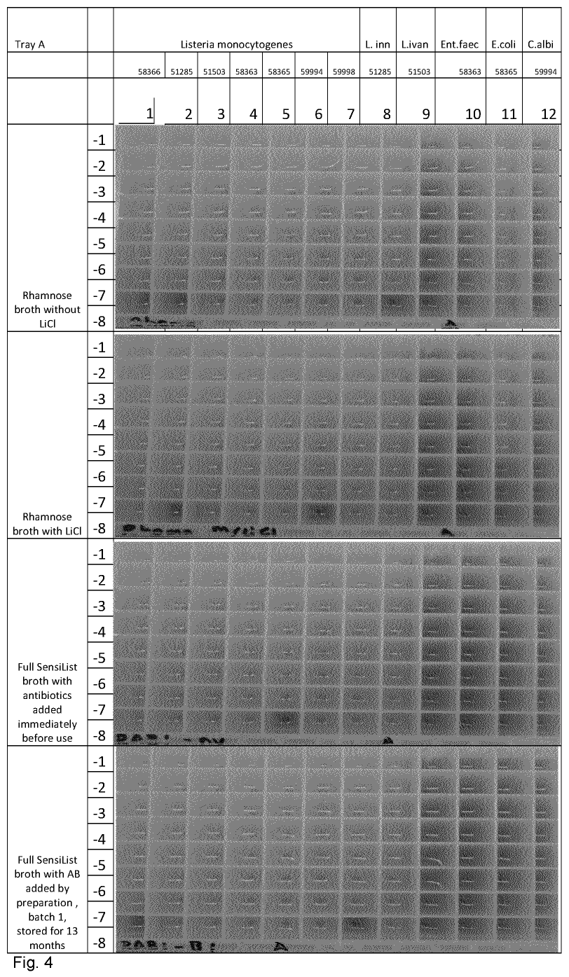

The sensitivity of the method was tested further in order to investigate whether homogenization of the whole salmon piece was needed, or whether shaking the samples with the SensiList broth was sufficient. One positive sample was mixed with 9 negative samples in order to obtain an accurate inoculation level with very low concentrations of L. monocytogenes (0.2-20 cfu/g). The theoretical concentration in the broth is 10 times lower, as sample and broth was mixed in the ratio 1:2.5. The results are shown in FIG. 3.

L. monocytogenes was detected with the method of the present document for all the inoculated samples, even for the lowest concentrations. The ISO method only detected the highest concentrations (20 cfu/g). After cultivation of the broth overnight, analysis with the ISO method detected all samples with concentration 2 cfu/g or higher, but only one of the three samples with 0.2 cfu/g, even though L. monocytogenes were detected with the method of the present document already immediately after the salmon pieces were shaken with the broth. The most likely explanation to this observation was that the only a small volume (0.1-1.0 ml of solution) is plated with the ISO method, while the entire broth is analysed with the method of the present document.

1.9.2 False Positive and Negative Bacteria

Similar experiments as described above were performed with different mixes of Listeria strains and salmon samples, but with an adapted version of the ISO method to obtain a higher sensitivity of it (see Materials and Methods). In all cases, there was a good correlation between methods, but also here, the method of the present document was found to be the most sensitive one. However, in samples with L. innocua, either alone or in a mix with L. monocytogenes the method of the present documents gave a higher concentration of presumptive positives than confirmed positives than the ISO method. This was due to that L. innocua is also able ferment rhamnose, and have similar tolerance to antibiotics and LiCl as L. monocytogenes. The confirmation step is therefore needed to separate L. monocytogenes from L. innocua.

Strains 1-18 were false positives obtained from salmon bought in fish delicatessen shops. The strains were identified with Malditoff technology as Hafnia alvei, Citrobacter sp, Aeromonas sp, Enterobacter cloacae, Alcaligenes faecalis, Eterococcus faecalis, Bacillus cereus and Macrococcus (the latter being uncertain). Strains 19-24 were L. monocytogenes. The conclusion was that the antibiotics tested were sufficient to avoid false positive results.

1.9.3 Stability of the SensiList Broth

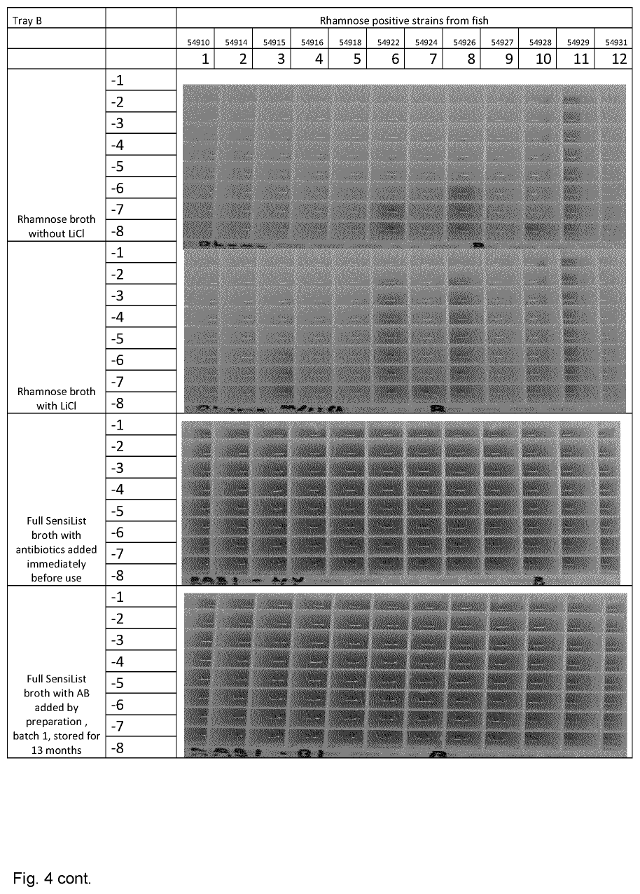

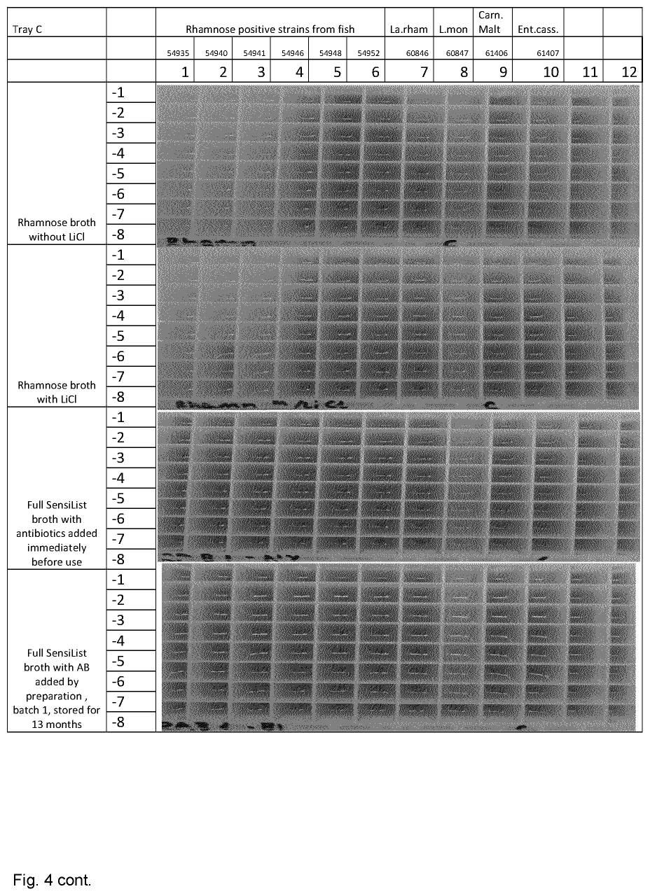

The test strains were cultivated in different formulations of the SensiList broth over a period of one year. The results after 48 hours incubation of the SensiList broth for all strains in fresh broth and broth stored for approximately one year before use are given below. All L. monocytogenes (tray A column 1-7 and Tray C column 8) strains and L. innocua (Tray A, column 8) strains gave positive results on all formulations, as expected. The other strains, however, gave false positive results only in the formulation with no LiCl and/or no antibiotics. All formulations with antibiotics, either the antibiotics were added immediately before use or from had been present in the broth for a year, gained correct results for all the three batches, indicating that the full broth is stable and precise. The results are presented in FIG. 4.

1.9.4 Confirmation of Presumptive Positive Results

In all experiments with the SensiList broth, plating on ALOA and the confirmation tests described in the ISO method yielded correct results.

Confirmation with PCR technology did also give correct result. Commercially available kits for DNA isolation gave in all the tested cases sufficient amount and quality of DNA to perform PCR analysis. The qPCR method applied is the same one as the one used to determine the genoserogroup, also called the molecular serogroup. The results for one of the strains below is presented in the table below. For all formulations of the SensiList broth, the same genes gave correct signals. The negative signals (marked as 0) are negative because the test strain is serogroup IIa.

Any other PCR method for detection of L. monocytogenes could have been used, but this method was chosen because it identifies both Listeria monocytogenes and the serogroup. Test of serogroup is normally performed in a separate analysis. Here, by using it as a confirmation step, confirmation and sufficient characterization to check out link to outbreaks or similarity to other isolates is obtained already in the primary analysis of the sample.

TABLE 5

| | | | | | | | Resultat/ | Correct |
|---|---|---|---|---|---|---|---|---|
| Sample | ORF2819 | ORF2110 | Imo1118 | Imo0737 | plcA | prs | serotype | data |
| BPV_L1 | 0 | 0 | 0 | 1 | 1 | 1 | IIa | IIa |
| HF_L1 | 0 | 0 | 0 | 1 | 1 | 1 | IIa | IIa |
| MQ_L1 | 0 | 0 | 0 | 0 | 0 | 0 | No | EBK |
| | | | | | | | listeria | |
| RAB1_L1 | 0 | 0 | 0 | 1 | 1 | 1 | IIa | IIa |
| RAB2_L1 | 0 | 0 | 0 | 1 | 1 | 1 | IIa | IIa |
| RAB3_L1 | 0 | 0 | 0 | 1 | 1 | 1 | IIa | IIa |
| RAB4_L1 | 0 | 0 | 0 | 1 | 1 | 1 | IIa | IIa |
| RhamnLiCl_L1 | 0 | 0 | 0 | 1 | 1 | 1 | IIa | IIa |
| Rhmamn_L1 | 0 | 0 | 0 | 1 | 1 | 1 | IIa | IIa |

### 2 Example 2: Demonstration of the Utility of the Method of the Present Document for Detecting Listeria in Food Samples

2.1 Case 1: Artificially and Naturally Contaminated Salmon

The studies described above are performed with salmon. The results were in all cases good.

In addition, a study with frozen and thawed salmon pieces were performed. The released thawing water was sampled with the ISO (ALOA) method and with the method of the present document. Two different mixes of L. monocytogenes were applied, inoculated in concentrations 2 and 20 cfu/g. For incubation, two temperatures were tested. The results are shown below in Table 5. Of all the analysed samples, the ISO method gave 6 negative results, while the method of the present document gave only one negative result in inoculated samples. All discrepancies were observed for the lowest inoculation level. Lower counts with increased frozen storage time of the fish appear to be due to reduction of alive bacteria during frozen storage.

The conclusion of this experiment was that the method of the present document is suited for detection and quantification of low concentrations of Listeria from fresh as well as frozen salmon.

TABLE 6

| | Storage scenario: | | | | | |
|---|---|---|---|---|---|---|
| | | | | Measured | | |
| | | | Measured | concentration of | Measured | |
| | | | concentration of | L. monocytogenes | concentration of | Measured |
| | | | L. monocytogenes | in released | L. monocytogenes | concentration of |
| | Listeria mix or | | in released | thawing water | in released | L. monocytogenes |
| | single strains, | Inoculation | thawing water | Modified | thawing water | in released |
| | incubation | concentration | ISO method | ISO method | SensiList | thawing water |
| | temperature with | in fish before | (cfu/g) | ALOA (cfu/g) | (cfu/g) | SensiList |
| | the method of the | freezing | (detection | (detection | Presumptive | confirmed with |
| | present document | (cfu/g) | limit 10 cfu/g) | limit 2 cfu/g) | positive | ALOA cfu/g) |
| Before storage | Not Inoculated, | 0 | 0 | 0 | 0 | 0 |
| | 37° C. | | 0 | 0 | 0 | 0 |
| | | | 0 | 0 | 0 | 0 |
| | Mix 1, 37° C. | 2 | 0 | 2 | 1 | 1 |
| | | | 0 | 2 | 2.2 | 2.2 |
| | | | 0 | 2 | 1.8 | 1.8 |
| | | 20 | 20 | 16 | 26 | 26 |
| | | | 10 | 10 | 48 | 48 |
| | | | 20 | 16 | 15.8 | 15.8 |
| | Mix 2, 37° C. | 2 | 0 | 4 | 1.4 | 1.4 |
| | | | 0 | 3 | 1 | 1 |
| | | | 0 | 3 | 1.4 | 0.8 |
| | | 20 | 20 | 16 | 9.2 | 4.2 |
| | | | 10 | 10 | 12.6 | 12.6 |
| | | | 10 | 12 | 9.8 | 9.8 |
| | Not Inoculated, | 0 | 0 | 0 | 0 | 0 |
| | 30° C. | | 0 | 0 | 0 | 0 |
| | | | 0 | 0 | 0 | 0 |
| | Mix 2, 30° C. | 2 | 0 | 4 | 1.6 | 1.6 |
| | | | 0 | 3 | 2.2 | 1.6 |
| | | | 0 | 3 | 1.4 | 1.4 |
| | | 20 | 20 | 16 | 44.2 | 44.2 |
| | | | 10 | 10 | 4.6 | 4.6 |
| | | | 10 | 12 | 9.8 | 9.8 |
| 1 week | Not Inoculated, | 0 | 0 | 0 | 0 | 0 |
| | 37° C. | | 0 | 0 | 0 | 0 |
| | | | 0 | 0 | 0 | 0 |
| | Mix 1, 37° C. | 2 | 0 | 1 | 0.4 | 0.4 |
| | | | 0 | 2 | 1 | 1 |
| | | | 0 | 1 | 0.4 | 0.4 |
| | | 20 | 20 | 16 | 28.2 | 28.2 |
| | | | 10 | 10 | 15.8 | 15.8 |
| | | | 20 | 16 | 15.8 | 15.8 |
| | Mix 2, 37° C. | 2 | 0 | 0 | 3.4 | 0.8 |
| | | | 0 | 2 | 0 | 0 |
| | | | 0 | 0 | 1 | 0.4 |
| | | 20 | 0 | 6 | 15.8 | 4.4 |
| | | | 10 | 6 | 15.8 | 15.8 |
| | | | 0 | 2 | 9.8 | 9.8 |
| | Not Inoculated, | 0 | 0 | 0 | 0 | 0 |
| | 30° C. | | 0 | 0 | 0 | 0 |
| | | | 0 | 0 | 0 | 0 |
| | Mix 2, 30° C. | 2 | 0 | 0 | 1.6 | 1.6 |
| | | | 0 | 2 | 1.4 | 1.4 |
| | | | 0 | 0 | 0.4 | 0.4 |
| | | 20 | 0 | 6 | 9.8 | 9.8 |
| | | | 10 | 8 | 15.8 | 15.8 |
| | | | 0 | 2 | 14 | 9.2 |
| 5 weeks | Not Inoculated, | 0 | 0 | 0 | 0 | 0 |
| | 37° C. | | 0 | 0 | 0 | 0 |
| | | | 0 | 0 | 0 | 0 |
| | Mix 1, 37° C. | 2 | 0 | 0 | 2.6 | 2.6 |
| | | | 0 | 2 | 0.8 | 0.8 |
| | | | 0 | 1 | 0.4 | <0.4 |
| | | 20 | 10 | 6 | 6.6 | 6.6 |
| | | | 20 | 22 | 4.6 | 4.6 |
| | | | 10 | 10 | 12.6 | 16.8 |
| | Mix 2, 37° C. | 2 | 0 | 0 | 2.2 | 1.4 |
| | | | 0 | 2 | 0 | 0.4 |
| | | | 0 | 3 | 1 | 1.6 |
| | | 20 | 0 | 6 | 9.8 | 6.6 |
| | | | 10 | 8 | 6.6 | 6.6 |
| | | | 10 | 12 | 4.6 | 4.6 |
| | Not Inoculated, | 0 | 0 | 0 | 0 | 0 |
| | 30° C. | | 0 | 0 | 0 | 0 |
| | | | 0 | 0 | 0 | 0 |
| | Mix 2, 30° C. | 2 | 0 | 0 | 2.2 | 2.2 |
| | | | 0 | 2 | <0.4 | <0.4 |
| | | | 0 | 3 | 2.2 | 1.4 |
| | | 20 | 0 | 6 | 6.6 | 6.6 |
| | | | 10 | 8 | 15.8 | 15.8 |
| | | | 10 | 12 | 6.6 | 6.6 |

2.2 Case 2: Contaminated Heat Treated Chicken Meat

Heat treated chicken meat were inoculated with L. monocytogenes and analysed with the method of the present document and a modified ISO (ALOA) with a lower dilution factor of the sample, leading to detection level 1 cfu/g. The purpose was to investigate, firstly, whether the chicken meat interfered with the SensiList broth and gave false positive results, and secondly, whether the concentration were correctly estimated. The results are given below.

The method of the present document yielded correct detection in all cases, while the modified ISO method gave one false negative result. This was most likely due to the higher sensitivity of method of the present document.

The conclusions were that heat treated chicken meat did not interfere with the method of the present document.

Also, the method of the present document gave precise enumeration compared with the modified ISO method, precise enumeration down to 0.2 cfu/g with quantitray, and precise detection down to 1-3 cfu/total sample, see Table 6.

TABLE 7

| | | Measured concentration of | | |
|---|---|---|---|---|
| | | L. monocytogenes | | |
| | | in heat treated chicken meat | | |
| | | ALOA (cfu/g), | | |
| | | adapted ISO | | |
| | Inoculation | method to | | |
| | concentration | obtain | SensiList | SensiList |
| | (cfu/g) in | detection | (cfu/g) | confirmed |
| | heat treated | level | Presumptive | with ALOA |
| | chicken meat | 1 cfu/g | positive | cfu/g) |
| Not | 0 | <1 | <0.4 | <0.4 |
| inoculated | | <1 | <0.4 | <0.4 |
| | | <1 | <0.4 | <0.4 |
| Inoculated | 0.9 | 2 | 2.6 | 2.6 |
| with L.m. | | 2 | 1 | 1 |
| | | <1 | 1 | 1 |
| | 9 | 10 | 6.6 | 6.6 |
| | | 14 | 15.8 | 15.8 |
| | | 18 | 15.8 | 26 |
| | 90 | 216 | 69.6 | 69.6 |
| | | 196 | 108.4 | 108.4 |
| | | 230 | 108.4 | 108.4 |

2.3 Case 3: Naturally Contaminated Meat and Milk Products, Processed Products from Several Animal Species

Naturally contaminated food samples were obtained from reference laboratories for L. monocytogenes in foods in Europe. The samples were frozen, and Listeria bacteria may have died off during the storage. However, the samples were used to test firstly, interference of the food matrix with the method of the present document, secondly, a interference with a normal background flora, and third, a enumeration level.

Also in this case, the modified ISO method was used in order to obtain a sufficiently low detection level to make comparison with the method of the present document useful. The results are shown in the table below.

In general, there was a good correlation between the modified ISO method. The method of the present document detected L. monocytogenes in one more sample (noodles with chicken meat) than the ISO method, probably due to the higher sensitivity. The measured concentration was 0.4 cfu/g.

No interference with the tested food matrixes leading to false positive or negative results with the Method of the present document were observed.

One of the samples contained L. welshimeri. This gave a false positive result with SensiList and on ALOA. The result indicates that false positive results may be obtained in both the present and the prior art methods for detection and enumeration of Listeria. However, L. weshimeri is a very rare Listeria spp in food compared to L. innocua and L. monocytogenes and the specificity of the method of the present document is considered to be high enough and at least as high as for the presently available methods.

The conclusions from this study were that the method of the present document yielded true results for naturally contaminated samples. No significant negative interference with complex food matrixes was observed. See Table 7.

TABLE 8

| | | | Result Method of the present document | | |
|---|---|---|---|---|---|
| | Concentration of | ALOA | | | Confirmed |
| | L. monocytogenes | plates | *Presumptive | | results of |
| | before freezing. | (1:2 cont.) | positive- | Confirmation on ALOA | L. monocytogenes |
| Matrix | cfu/g | cfu/g | colour of wells | plates, observations | cfu/g |
| Beef meat, raw, | Unknown | <2 | All red | No typical colonies | <0.2 |
| vacuumpacked | | | | | |
| Vacuum | <100 | <2 | All red | No typical colonies | <0.2 |
| packed paté | | | | | |
| with meat | | | | | |
| Sausage of | >4500 | 3300 | All yellow | Some presumptive | 81 |
| pork meat, | | | | presumptive positives | |
| vacuum packed | | | | gave colonies with and | |
| | | | | some without | |
| | | | | characteristic zone. A | |
| | | | | mix of L. monocyotenes | |
| | | | | and L. innocus. | |
| Sausage of | >4500 | 90000 | All yellow | Typical colonies in most | >484 |
| pork meat, | | | | wells | |
| vacuum packed | | | | | |
| Cooked | 10 | 6 | 17of 96 large | Green colonies in all | <0.2 |
| pork meat | | | wells yellow, | yellow wells, but not | |
| | | | 1 of 96 small | with typical zones. | |
| | | | wells yellow | The colonies were | |
| | | | | L. welshimeri. | |
| | | | | No red wells contained | |
| | | | | typical colonies | |
| Noodles with | 173 | <10 | Some range | Typical colonies | 0.4 |
| chicken meat | | | wells | | |
| Pork meat, | 336 | 160 | Yellow wells: | All yellow wells had | 82 |
| raw | | | All large | typical colonies with | |
| | | | 21 small | zones | |
| Calf meat, | 10 | <2 | All red | No typical colonies | <0.2 |
| raw | | | | | |
| Goat cheese, | >4500 | 1000 | All yellow | Typical colonies in all | >484 |
| unpasteurised | | | | yellow wells | |
| Sushi | <10 | <2 | All red | No typical colonies | <0.2 |
| Raw sausage | <10 | <2 | All red | No typical colonies | <0.2 |
| with pork meat | | | | | |
| Raw chicken | <10 | <2 | 1 yellow well | Yellow well contained | <0.2 |
| meat | | | | L. innocus | |
| Raw beef | <10 | <2 | All red | No typical colonies | <0.2 |
| meat | | | | | |

2.4 Case 4: Swabs for Testing of Production Surfaces after Cleaning

Swabs from testing of production equipment surfaces were obtained from three factories. Such swabs are supplied with a neutralizing solution. The test was performed in order to investigate whether the material, the neutralizing solution or residuals from cleaning solutions could interfere with the method of the present document, for instance by introducing growth inhibiting compounds or increased buffer capacity which would both lead to possible false negative results.

The samples obtained from the companies were inoculated with L. monocytogenes 0, 15 or 1500 cfu/swab. The swabs were placed in SensiList broth, and the solution transferred to a quantitray and incubated as described above.

None of the unioculated swabs gave presumptive positive results. For swabs inoculated with 15 cfu of L. monocytogenes, 4-9 of 96 wells turned yellow and were confirmed positive for L. monocytogenes. For swabs incolated with 1500 cfu, 58-96 wells turned yellow. In a few cases, some wells changed from red to orange, but not to yellow. The colour change was less than qualified for a presumptive positive result.

The conclusion from the study was that the method of the present document yielded correct results for detection of L. monocytogenes in swabs used for production environment sampling and the enumeration is sufficient.

2.5 Case 5. Test of Production Samples in a Salmon Production Company

A salmon processing company took samples of process water and tampoons from “sluk” and inserted in SensiList broth. The samples were incubated in the company, and sent for confirmation at a local lab. The results after the first cultivation are given in FIG. 4.

According to the company, the sampling and reading of colour change was easy. Confirmation of the samples showed that the yellow flasks contained L. innocua, while the red flasks did not contain Listeria spp.

The water and “sluk”/drainage samples were taken with water absorbing (tamponger). These did not give any interference.

The salmon samples were 100 g each.

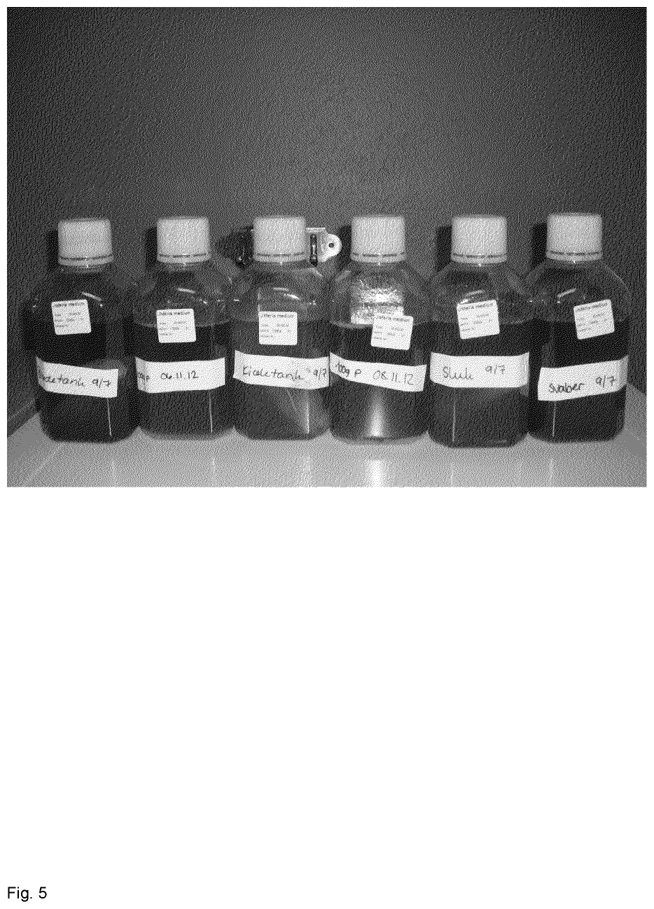

The conclusion from the study, presented in FIG. 5, was that SensiList is sufficient user friendly for use in a company. Presumptive positive results matched the results from the lab.

### Example 3: Comparison of Food Grade Rhamnose Compared with Analytical Grade of Rhamnose

Rhamnose is a rare carbohydrate and relatively expensive. As the first culture medium according to the present document uses this component and which has a high volume, this component will have a large impact on the price of the method/kit. In order to check if a food grade rhamnose could be used instead of an analytical grade rhamnose, the SensiList broth was prepared in two versions and compared. The comparison was carried out in two large trials:
1. With single cell cultures with the same strains used in the stability test of the broth. The qualitative method was applied.
2. With naturally contaminated and not contaminated samples from two fish slaughter and processing companies. The samples were water, wet swabs, dry swabs and fish flesh. Both the qualitative and the quantitative method was applied. After cultivating in the Sensilist broth, three agar media were used for confirmation: Blood agar. Rapid L'mono and ALOA for confirmation.

Results

The test of single stains gave in all cases correct identification both with the analytical grade rhamnose and the food grade rhamnose.

For the samples from the fish production facilities, there was good correlation between samples, meaning that the positive and negative results were positive for both the food grade and analytical grade rhamnose. However, there were deviations. This was most likely due to that the concentrations of L. monocytogenes were very low, i.e. never above 6 cfu/total sample. With such low concentrations, if is random in which of the two broths the Listeria was transferred to, which in turn means that only the broth with the Listeria could possible give a positive result.

An overview of results from one company, samples taken over time is given below. The crosses indicate negative samples, while the Pos means detected. All concentrations were very low. A previous amendment of the ISO method, using less diluent and thereby obtain a detection level for enumeration of 2 cfu/g is not sufficient to detect Listeria in these samples.

Samples from company Positive samples: Never more than 3 positive wells: indicate <6 cfu/sample

TABLE 9
The table is an example from use in industry. The Norwegian text in the leftmost column shows positions
in the production line. The top row shows dates for samples, where “Pos” denotes a positive
sample. The rightmost column has comments to the tests, such as “swab of 10 filets”, “meat samples”, “water”.

| | 19.aug | 20.aug | 21.aug | 22.aug | 23.aug | 26.aug | 27.aug | 28.aug | 29.aug | 30.aug | |
|---|---|---|---|---|---|---|---|---|---|---|---|
| File L3 | x | x | x | Pos | x | x | | | x | | Swab av 10 filet |
| File L3 | x | x | x | x | x | x | | | x | | Kjøtt prøve |
| Etter BA581, L3 | | x | Pos | x | Pos | x | | | x | | Miljøprøver |
| Baader 581, L3 | | Pos | Pos | Pos | x | x | | | x | | 2 timer samling |
| File L1 | | x | x | x | x | | x | x | | | Swab av 10 filet |
| File L1 | | x | x | x | x | | x | x | | | Kjøtt prøve |
| Etter BA581, L1 | | x | x | x | x | | x | x | | | Miljøprøver |
| Baader 581, L1 | | x | x | x | x | | x | x | | | 2 timer samling |
| Manuell sløyebord | | | x | x | x | | Pos | x | x | | 2 timer samling |
| Baader 142, 7 + 8 | | | Pos | Pos | Pos | Pos | Pos | x | x | | 2 timer samling |
| Baader 142, 5 | | | x | Pos | x | x | Pos | x | x | | 2 timer samling |
| Baader 142, 2 | | | | | | x | Pos | x | x | | 2 timer samling |
| Vann ut fra Baader 8 | | | | | | | | x | x | | Vann |
| Vann ut fra Baader 5 | | | | | | | | x | x | | Vann |
| Vann ut fra Baader 2 | | | | | | | | x | x | | Vann |
| Etterkjølingstank | | | | | | | | x | x | | |

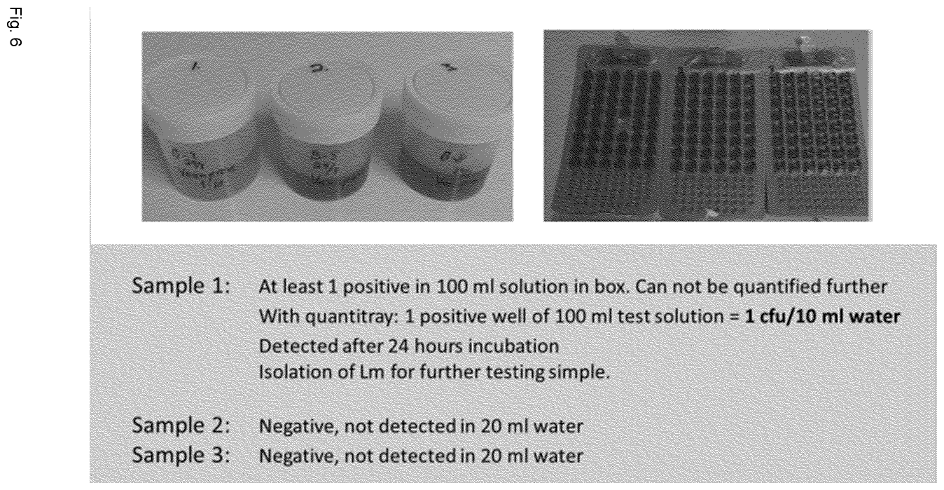

The results for three of the samples are given in FIG. 6. The boxes to the left represent the qualitative method, where 1 L. monocytogenesin the sample, in this case 20 mi of water, can be detected. Only the sample to the left was positive. The same samples were analysed with the quantitative method (right panel). In this case, 100 ml of sample (10 ml of water and 90 ml SensiList broth) were transferred to a quantitray. Only one well changed colour from red to yellow, which means that there was the most likely concentration of L. monocytogenes in the water was 1 cfu/10 ml. The result was obtained after 24 hours of incubation.

The SensiList method (i.e. the method of the present document) worked well for all samples, but the dilution fraction needs to be considered in particular for very wet samples and for fish meat. This is, on one hand, due to dilution of the SensiList broth, as the pH is related to the amount of acid produced, and thereby the concentration of rhamnose. On the other hand, a high relative amount of other carbon and energy sources than rhamnose, like fish flesh, may lead to that the bacteria grow on these instead of rhamnose, and the sufficient amount of acid will not be produced. Both these challenges can easily be compensated for in the SensiList broth if a sufficiently high fraction of broth is used.

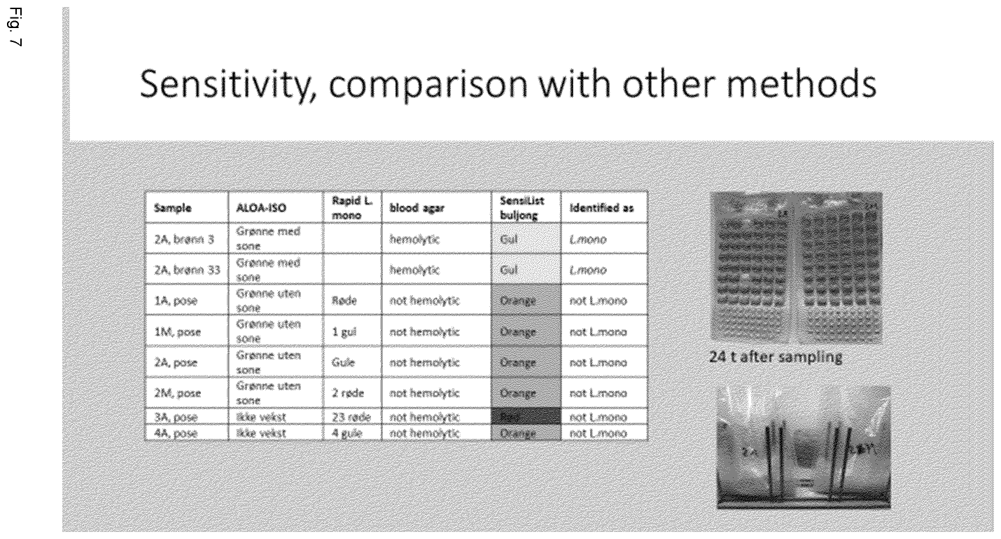

Results from a presumptive positive sample is given in FIG. 7. Two wells were yellow, a few orange and most wells red. Material from each well were transferred to three different growth media in order to test specificity. Only the yellow wells gave hemolysis on blood agar and typical zones for L. monocytogenes on ALOA agar. The orange and red wells gave either no growth or growth of bacteria not being L. monocytogenes. Thus, the sensitivity and selectivity of the SensiList with the rhamnose broth was as good as previously found with analytical grade rhamnose.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Unless expressly described to the contrary, each of the preferred features described herein can be used in combination with any and all of the other herein described preferred features.

### REFERENCES

Vitullo et al., Real-time PCRs assay for serogrouping Listeria monocytogenes and differentiation from other Listeria spp., Molecular and Cellular Probes, Volume 27. Issue 1, February 2013, Pages 68-70.
Bjørn C. T. Schirmer, Solveig Langsrud, Trond Møretrø, Therese Hagtvedt, Even Heir, 2012. Performance of two commercial rapid methods for sampling and detection of Listeria in small-scale cheese producing and salmon processing environments. Journal of Microbiological Methods, Volume 91, Issue 2, Pages 295-300, ISSN 0167-7012, https://doi.org/10.1016/j.mimet.2012.08.013

1. A method for the detection of rhamnose fermenting Listeria spp., such as Listeria monocytogenes, in a sample, said method comprising or consisting of the steps of:
i) preparing a suspension of a sample potentially containing Listeria bacteria in a first culture medium, said first culture medium comprising rhamnose, one or more of an antibiotic, a pH colour indicator and LiCl;
ii) incubating said suspension at conditions allowing the growth of Listeria bacteria; and
iii) identifying positive samples.

2. A method according to claim 1, said method further comprising the enumeration of rhamnose fermenting Listeria spp., said method comprising a step ia) of transferring the suspension prepared in step i) to a multi-well tray and a step iv) being performed after step iii) of calculating the concentration of said Listeria in said sample, such as by using the Most Probable Number method.

3. The method according to claim 1 or 2, further including a step of confirming the presence of Listeria monocytogenes, such as by plating positive samples identified in step iii) on a second growth medium such as ALOA medium, by utilizing a molecular method, such as by polymerase chain reaction or in situ hybridization, ELISA, VITEC, and/or API, said step being performed after step iii) in claim 1 or step iv) in claim 2.

4. The method according to any one of the preceding claims, wherein said sample possibly containing rhamnose fermenting Listeria spp. is obtained by dividing said sample into small pieces such as by homogenization, slicing and/or chopping either before step i) is performed and/or in the first culture medium of step i).

5. The method according to any one of the preceding claims wherein said antibiotic is one or more of nalidixic acid, ceftazidime, polymyxin B sulfate, cycloheximide, amphotericin B, preferably a combination of two or more of any of said antibiotics.

6. A method according to any one of the preceding claims wherein said pH colour indicator is phenol red.

7. A method according to any one of the preceding claims wherein said LiCl is present in an amount of about 5-17 g/l, such as about 7-13 g/l, such as about 10 g/l.

8. A method according to any one of the preceding claims wherein said rhamnose is present in an amount of about 5-17 g/l, such as about 7-13 g/l, such as about 10 g/l.

9. The method according to any one of the preceding claims, wherein said sample is a food sample, an environmental sample, or a sample from an animal, such as a human, such as a tissue sample or a feces sample.

10. The method according to any one of the preceding claims, wherein said food sample is raw or processed meat, poultry or fish products, vegetables or a ready-to-eat food product.

11. The method according to any one of claims 1-9, wherein said environmental sample is a water sample, a dirt sample or a food industry environmental sample, such as a surface swab sample.

12. The method according to any one of the preceding claims, wherein said method is performed in a closed system.

13. A culture medium for the growth and/or detection of rhamnose fermenting Listeria spp., such as Listeria monocytogenes, said culture medium comprising:
e) rhamnose in a concentration of about 5-15 g/l, such as about 7-13 g/l, such as about 10 g/l;
f) one or more antibiotics, such as nalidixic acid, ceftazidime, polymyxin B sulfate, cycloheximide, amphotericin B, preferably at least two of these;
g) a pH colour indicator, such as phenol red; and
h) LiCl in a concentration of about from 5 to about 15 g/l, such as about from 7 to about 13 g/l, such as about 10 g/l.

14. A kit for detecting and/or enumerating rhamnose fermenting Listeria spp., such as Listeria monocytogenes, in a sample, said kit comprising:
a) a container comprising a culture medium comprising or consisting of rhamnose, one or more of an antibiotic, a pH colour indicator and LiCl;
b) a container or multi-well tray for cultivating a sample possibly containing Listeria bacteria; and
c) optionally a colour chart for identifying positive samples; and
d) optionally instructions for use.

15. A computer implemented calculator for displaying a prediction of the growth of Listeria, such as Listeria monocytogenes, based on the detection and enumeration of colony forming units in a sample by the method according to any one of claims 1-12, said calculator comprising:
c) an input device, such as a keyboard or a microphone;
b) an output device, such as a display, a computer or mobile phone screen or a loudspeaker;
d) software, downloadable or in memory, such as an app on a smart phone or a webpage;
where the software accepts the input of any number of colony-forming units found in the sample in the range of 0.04 cfu/g to 1 cfu/g.
